(19) United States
(12) Patent Application Publication
(10) Pub. No.: US 2016/0002667 A1
(43) Pub. Date: Jan. 7, 2016

(54) PSEUDOMONAS EXOTOXINS FOR CANCER TREATMENT

(71) Applicant: RUSH UNIVERSITY MEDICAL CENTER, Chicago, IL (US)

(72) Inventors: Howard Kaufman, Chicago, IL (US); Carl Ruby, Oak Park, IL (US); Sasha Shafikhani, Oakland Park, IL (US)

(21) Appl. No.: 14/767,105

(22) PCT Filed: Mar. 10, 2014
(86) PCT No.: PCT/US14/22499

§ 371 Date: Aug. 11, 2015

Related U.S. Application Data
(60) Provisional application No. 61/792,606, filed on Mar. 15, 2013.

(51) Int. Cl. C12N 15/86; A61K 38/45; C12N 9/10; A61K 45/06

(52) U.S. Cl. CPC C12N 15/86; A61K 45/06; A61K 38/45; C12N 9/1048; C12N 2710/24143

(57) ABSTRACT

The invention generally relates to recombinant nucleic acid constructs that comprise a nucleotide sequence encoding Pseudomonas aeruginosa exotoxin. The invention also relates to the use of Pseudomonas aeruginosa exotoxins for treating cancer.

### RELATED APPLICATIONS

This application claims the benefit of U.S. Patent Application No. 61/792,606 filed on Mar. 15, 2013, the entire teachings of which are incorporated herein by reference.

### BACKGROUND OF THE INVENTION

Cancer is the second leading cause of death in industrial countries. Treatments for most patients often include a combination of surgery, chemotherapy, hormone therapy and/or ionizing radiation. In general, these treatments are at least partially effective at the beginning, however, after a variable period of time, progression occurs. Many cancers show initial or compulsory chemo-resistance. Resistance to cytotoxic agents used in cancer therapy remains a major obstacle in the treatment of human malignancies. Since most anti-cancer agents were discovered through empirical screens, efforts to overcome resistance are hindered by our limited understanding of why these agents are effective.

The pathogenic bacterium Pseudomonas aeruginosa can to cause severe acute and chronic infections in humans. Pseudomonas exotoxin A (PE) is the most toxic virulence factor of this bacterium. It has ADP-ribosylation activity and affects the protein synthesis of the host cells. The cytotoxic pathways of PE have been elucidated, and it has been shown that PE uses several molecular strategies developed under evolutionary pressure for effective killing. Interestingly, a medical benefit from this molecule has also been ascertained, and several PE-based immunotoxins have been constructed and tested in preclinical and clinical trials against different cancers. In these molecules, the enzymatically active domain of PE is specifically targeted to tumor-related antigens. See, e.g., Wolf et al., Pseudomonas exotoxin A: from virulence factor to anti-cancer agent. Int J Med Microbiol. 2009 March; 299(3):161-76. doi: 10.1016/j.ijmm.2008.08.003.

Despite significant progress in recent years, cancer remains amongst the deadliest diseases worldwide, particularly metastatic cancer. A need exists for developing new cancer therapies.

### SUMMARY OF THE INVENTION

The invention generally relates to recombinant nucleic acid constructs that comprise a nucleotide sequence encoding a Pseudomonas aeruginosa exotoxin. The invention also relates to the use of Pseudomonas aeruginosa exotoxins for treating cancer.

### BRIEF DESCRIPTION OF THE DRAWINGS

FIGS. 1A and 1B show the cytotoxic effect of Pseudomonas aeruginosa exotoxins ExoU, ExoS, and ExoT. Pseudomonas aeruginosa exotoxins are more effective than cisplatin in killing 4T1 metastatic breast cancer cells.

FIG. 2 shows that current cancer drugs have limited effect on different breast cancers. The breast cancer cell lines 4T1, MCF-7, EMT6 and MDA-MB-231 were treated with the chemotheraputic drugs paclitaxel (5 uM), 5-fluorouracil (1 uM), cisplatin (50 uM), 4-hydroxytamoxifen (1 uM), doxorubicin (5 uM) or vehicle alone (DMSO). Cells were treated in the presence of the impermeant dye propidium iodide (PI) which fluoresces red when cells dye and observed by time-lapse videomicroscopy. Cytotoxicity was determined by measuring the PI fluorescence intensity at each time point using ImageJ (NIH).

FIG. 3 shows that Pseudomonas exotoxins demonstrated greater cytotoxicity than cisplatin treatment. 4T1 cells were infected with Pseudomonas aeurigonsa strains expressing either ExoU, ExoS, or ExoT or treated with cisplatin (50 uM). The cell death stain propidium iodide (PI) was added and the cells were observed by time-lapse videomicroscopy. Cytotoxicity was determined by measuring the PI fluorescence intensity at each time point using ImageJ (NIH).

FIG. 4 is a table summarizing the effect of Pseudomonas exotoxins on various cancer cell lines. Pseudomonas exotoxins are able to kill a variety of cancer cell lines. Listed cell lines were infected with Pseudomonas aeruginosa expressing either ExoU, ExoS, or ExoT in the presence of the cell death dye propidium iodide and observed by time-lapse videomicroscopy. Based on PI staining analysis, the mean time to death was determined for each cell line. Note that ExoU and ExoS showed the fastest killing and greatest potentcy while ExoT was less potent and unable to kill some cell lines. (ND: no death).

FIG. 5 shows that the ADPRT domain of ExoT is primarily responsible for ExoT-mediated cytotoxicity in B16 and A375 cells. B16 and A375 cells were transfected with either GFP-fused ExoT, GAP and/or ADPRT domain mutants or GFP vector alone. Cytotoxicty was determined by positive propidium iodide staining. ExoT and the ADPRT domain show significantly greater cytotoxicity than the GAP domain alone, the GAP/ADPRT double mutant or GFP alone for both B16 and A375 cells (p<0.05).

FIG. 6 shows that ExoT causes cells to arrest in G1. B16 cells were transfected with GFP vector, Exot-GFP or left untransfected. Following 24 h of transfection, cells were analyzed by FACS for the cell cycle stage of transfected cells. The graph shows the percentage of cells in G1, S, or G2. Note the increase in cells in G1 and decrease in S and G2 for pExoT transfected cells compared to untransfected and GFP alone.

FIG. 7 shows that cell cycle arrest is mediated by both GAP and ADPRT domains of ExoT. A375 cells were transfected with ExoT, the GAP or ADPRT domain mutants or GFP vector alone. Cells were observed by time-lapse video microscopy. The number of mitotic events per cells in each field of view was counted. ExoT showed a complete block of mitotic cells, while the ADPRT or GAP domain showed about 3-5% mitotic cells. This is significantly less than the ˜33% of mitotic cells observed with the vector alone.

FIG. 8 shows the ExoT-mediated cell cycle arrest of A549 lung and MCF7 breast cancer lines. A549 cells transfected with GFP, ExoT-GFP, or left untransfected were observed by time-lapse videomicroscopy. The percentage of cell divisions occurring during a 5 h time period shows now divisions for ExoT-GFP transfected cells as compared to the 8-10% observed for untransfected or vector alone.

FIG. 9 shows that ExoT inhibits B16 motility. B16 cells were transfected with ExoT, the GAP or ADPRT domain mutants or GFP vector alone. Cells were observed by time-lapse video microscopy and tracked over a 10 h period using ImageJ (NIH). The mean track length during this time is shown. ExoT and the ADPRT and GAP domain mutants show significantly shorter track lengths than GFP transfected cells. Importantly, ExoT could have an impact on reducing metastasis of cancer cells.

FIG. 10 shows that ExoT is capable of inducing cell death through multiple pathways. ExoT has been shown to block cytokinesis as well as arrest cells in G1. ExoT is also able to induce early apoptotic cell death by inducing anoikis as well as more delayed necrotic type killing by inhibiting phospho-glycerate kinase-1 through ADPRT. These features make ExoT an excellent candidate as a novel cancer therapeutic.

### DETAILED DESCRIPTION OF THE INVENTION

1. Overview

The invention generally relates to recombinant nucleic acid constructs that comprise a nucleotide sequence encoding a Pseudomonas aeruginosa exotoxin. The invention also relates to the use of Pseudomonas aeruginosa exotoxins for treating cancer.

Treatments for most cancer patients include a combination of surgery, chemotherapy, hormone therapy and ionizing radiation. Resistance to cancer therapy is not only common but expected. The reasons for the failure of current therapies include but are not limited to: (i) therapy having limited cellular targets; (2) therapy targeting biosynthetic cellular processes for which physiological responses are present; (3) drug resistance pumps; (4) therapy's failure to induce an effective anti-tumor immune response; and (5) the induction of apoptotic compensatory proliferation signaling.

As disclosed and exemplified herein, Pseudomonas aeruginosa exotoxins (in particular, ExoT, ExoU, and ExoS) possess unique properties that make them good candidates to be used, alone or in combination, to treat cancer (e.g., eradicating breast cancer metastases). Pseudomonas aeruginosa exotoxins offer several advantages, as compared current cancer treatment regimen. First, Pseudomonas aeruginosa exotoxins are highly potent inducers of cell death. Second, it is much more difficult for cancer cells to develop resistance to Pseudomonas exotoxins because they target multiple cellular proteins and cellular processes that function in cell proliferation and survival. Third, unlike current therapies that primarily induce apoptosis, a type of cytotoxicity which is generally believed to be anti-inflammatory in nature, Pseudomonas exotoxin-mediated cytotoxicities have been shown to induce a strong pro-inflammatory environment. Fourth, the inventors discovered that Pseudomonas exotoxins (such as Exotoxin T) can block a previously unknown pathway of cancer resistance that could be a major contributor to ineffectiveness of therapy in the treatment of cancer, in particular breast cancer. Finally, Pseudomonas exotoxins (such as Exotoxin T) are also potent anti-proliferative agents.

The invention also relates to the delivery of Pseudomonas exotoxins to cancer cells. In particular, viral vectors encoding an exotoxin can be used to deliver an exotoxin to a cancer cell. A preferred viral vector is a Vaccinia virus vector.

2. Definitions

As used herein, the singular forms “a,” “an” and “the” include plural references unless the content clearly dictates otherwise.

The term “about”, as used here, refers to +/−10% of a value.

The term “recombinant viral vector” refers to a recombinant nucleic acid construct comprising a sequence that encodes a non-viral protein and one or more sequences encoding viral expression control elements and/or viral proteins. Typically certain portion(s) of the native viral genome has (have) been removed and/or modified, such that the viral vector can be used to carry one or more exogenous nucleic acid sequences, and deliver the exogenous nucleic acid sequence(s) to a host cell. A recombinant viral vector may be replication-deficient, or it may be capable of replication, for example when associated with the proper control elements. The recombinant viral vectors described herein comprises a nucleic acid sequence encoding a Pseudomonas aeruginosa exotoxin that, preferably is operable liked to a control element such that the exotoxin can be produced in a cancer cell. A recombinant viral vector may be DNA, RNA or contain DNA and RNA.

The term “replication deficient” or “replication defective” refers to a viral genome that does not comprise all the genetic information necessary for replication and formation of a genome-containing capsid in a replication competent cell under physiologic (e.g., in vivo) conditions.

As used herein, the term “operably linked” refers to a first polynucleotide sequence, such as a promoter, connected with a second polynucleotide sequence, such as a coding sequence of interest, where the polynucleotide molecules are arranged so that the first polynucleotide sequence affects the function of the second polynucleotide sequence. For example, a promoter that is operably linked to a coding sequence of interest can modulate transcription of the coding sequence in a cell.

3. Cytotoxins of Pseudomonas Aeruginosa

Exotoxins from any strains of Pseudomonas aeruginosa can be used for the invention. Preferred exotoxins are ExoT, ExoU, and ExoS. Pseudomonas aeruginosa has many strains, including, for example, Pseudomonas aeruginosa strain PA01, Pseudomonas aeruginosa PA7, Pseudomonas aeruginosa strain UCBPP-PA14, and Pseudomonas aeruginosa strain 2192.

Pseudomonas aeruginosa produces exotoxin A (ETA) and four type III cytotoxins: ExoS, ExoT, ExoU and ExoY. Different clinical isolates of P. aeruginosa can express one or more of these four exotoxins. Each of ETA, ExoS, ExoT, ExoU and ExoY has a well-characterized enzymatic activity that is important for cytotoxicity. The catalytic activity of each type III cytotoxin is activated by a host protein.

ETA is the most potent protein toxin that P. aeruginosa secretes, and it inhibits mammalian protein synthesis by ADP-ribosylation of elongation factor 2 (EF2).

ExoS ADP ribosylates the early host proteins ezrin, radixin and moesin proteins (ERMs). ERMs are believed to be functionally redundant and have roles in cell shape, microvilli, motility, cell adhesion and phagocytosis, and at an important step in actin regulation. ADP-ribosylation might induce cytoskeletal rearrangements by disrupting the interaction of ERMs with Rho-signalling through the non-activation of the Rho-GDI (guanine nucleotide dissociation inhibitor) complex, or by interfering with the interaction of actin with focal adhesions. Therefore, in the early stages of P. aeruginosa infection, the ADP-ribosylation of ERMs disrupts the actin cytoskeleton and phagocytic capacity of the target cell in conjunction with the expression of Rho GAP activity.

ExoT has also shown to ADP-ribosylate Crk proteins. The ADP-ribosylation of Crk implicates the inactivation of Rac1 in the integrin signalling pathway, and implies that ExoT interferes with cell migration (wound healing) or phagocytosis; two Rac1-dependent functions.

Both ExoS and ExoT inactivate Rho GTPases directly by Rho GAP activity, and indirectly by ADP-ribosyltransferase activity. These toxins use both non-covalent (Rho GAP activity) and covalent (ADP-ribosylation) mechanisms to inactive the actin cytoskeleton.

ExoS and ExoT are closely related bifunctional proteins (74% identity at the amino acid level). ExoS has been extensively studied. Its N-terminal domain possesses an arginine finger motif characteristic of GTPase activating proteins (GAPs). ExoS exhibits GAP activity towards Rho, Rac, and Cdc42 in vitro and in vivo and has been shown to be sufficient to disrupt the actin cytoskeleton. Its GAP domain is an example of an expanding group of bacterial GAPs that appear to have arisen by convergent evolution.

The C-terminal domain of ExoS possesses ADP ribosyltransferase (ADPRT) activity towards Ras, Ral, and various Rab family GTPases and requires a eukaryotic 14-3-3 protein, factor-activating exoenzyme S, for activity. This activity interferes with eukaryotic DNA synthesis and endocytosis and causes cytotoxicity and cell death of mammalian cells. In vitro, ExoS auto-ADP ribosylates the critical arginine residue (arginine 146) in its GAP domain, leading to downregulation of its GAP activity.

Like ExoS, ExoT has N-terminal GAP activity in vitro and in vivo towards Rho, Rac, and Cdc42. This activity contributes to (i) disruption of the actin cytoskeleton, resulting in cell rounding (but not cytotoxicity), (ii) prevention of bacterial internalization through its inhibition of Rho family GTPases, and (iii) inhibition of wound healing. The C terminus of ExoT appears to possess minimal ADPRT activity in vitro (0.2% compared to ExoS) toward a synthetic substrate in vitro. This finding potentially implies that it is nonfunctional in vivo, although the use of a synthetic substrate may have underestimated the catalytic activity of the ADPRT domain. Mutation of the invariant arginine of the ExoT GAP domain to lysine (R149K) resulted in complete loss of GAP activity in vitro but only partial diminution of anti-internalization and cell rounding activity without affecting efficiency of translocation. These findings suggested that the ADPRT domain contributes to the biological activity of ExoT. See, e.g., Garrity-Ryan et al., The ADP ribosyltransferase domain of Pseudomonas aeruginosa ExoT contributes to its biological activities, Infect Immun. 2004 January; 72(1):546-58.

ExoU is a lipase that disrupts membrane function in mammalian cells. ExoU is a 687-residue protein that, once translocated through the type III secretion systems (T3SS), induces cytotoxic effects leading to rapid necrotic cell death. The crystal structure of ExoU has been solved. See, e.g., Gendrin et al., Structural Basis of Cytotoxicity Mediated by the Type III Secretion Toxin ExoU from Pseudomonas aeruginosa. PLoS Pathog 8(4): e1002637. doi: 10.1371/journal.ppat.1002637.

ExoY is an adenylate cyclase that elevates intracellular cyclic AMP (cAMP) to supra-physiological levels, which indirectly disrupts the actin cytoskeleton. The sequence and functional studies of ExoY (e.g., active site of ExoY) have also been reported. See, e.g., Yahr et al., ExoY, an adenylate cyclase secreted by the Pseudomonas aeruginosa type III system, PNAS Nov. 10, 1998 vol. 95 no. 23 13899-13904.

Exemplary nucleotide and amino acid sequences of ExoU, ExoT, and ExoS are provided herein (SEQ ID NOs. 1-6).

Cytotoxic fragments of the exotoxins described herein may also be used. A cytotoxic fragment of an exotoxin comprises a portion, but no the full-length sequence of the exotoxin, while retaining the cytotoxicity.

The exotoxins or exotoxin fragments of the invention can be a naturally occurring protein which has cytotoxic activity, or an active variant of a naturally occurring protein.

As used herein, “active variants” refers to variant peptides which retain cytotoxic activity. An active variant differs in amino acid sequence from a reference exotoxin (such as SEQ ID NOs. 2, 4, 6) but retains cytotoxic activity. Active variants of exotoxins or exotoxin fragments include naturally occurring variants (e.g., allelic forms) and variants which are not known to occur naturally.

In general, cytotoxic fragments and variants of P. aeruginosa exotoxins retain the well-known enzymatic activity of the full-length exotoxin. For example, a cytotoxic fragment or variant can contain the enzymatically active domain of ExoU, ExoT or ExoS.

Generally, differences are limited so that the sequences of the reference polypeptide and the active variant are closely similar overall and, in many regions, identical. An active variant of an exotoxin or exotoxin fragment and a reference exotoxin or exotoxin fragment can differ in amino acid sequence by one or more amino acid substitutions, additions, deletions, truncations, fusions or any combination thereof. Preferably, amino acid substitutions are conservative substitutions. A conservative amino acid substitution refers to the replacement of a first amino acid by a second amino acid that has chemical and/or physical properties (e.g., charge, structure, polarity, hydrophobicity/hydrophilicity) which are similar to those of the first amino acid. Conservative substitutions include replacement of one amino acid by another within the following groups: lysine (K), arginine (R) and histidine (H); aspartate (D) and glutamate (E); asparagine (N), glutamine (Q), serine (S), threonine (T), tyrosine (Y), K, R, H, D and E; alanine (A), valine (V), leucine (L), isoleucine (I), proline (P), phenylalanine (F), tryptophan (W), methionine (M), cysteine (C) and glycine (G); F, W and Y; C, S and T.

Preferably, an active variant of an exotoxin shares at least about 85% amino acid sequence similarity or identity with a naturally occurring exotoxin (e.g., SEQ ID NO:2, SEQ ID NO:4, or SEQ ID NO: 6), preferably at least about 90% amino acid sequence similarity or identity, and more preferably at least about 95%, at least about 96%, at least about 97%, at least about 98%, or at least about 99% amino acid sequence similarity or identity with said exotoxin. Preferably, the percentage of identity is calculated over the full length of the active variant.

In certain embodiments, the active variant comprises fewer amino acid residues than a naturally occurring exotoxin. In this situation, the variant can share at least about 85% amino acid sequence similarity or identity with a corresponding portion of a naturally occurring exotoxin, preferably at least about 90% amino acid sequence similarity or identity, and more preferably at least about 95%, at least about 96%, at least about 97%, at least about 98%, or at least about 99% amino acid sequence similarity or identity with a corresponding portion of said exotoxin.

Portions of the amino acid sequence of an exotoxin which correspond to a variant and amino acid sequence similarity or identity can be identified using a suitable sequence alignment algorithm, such as ClustalW2 (http://www.ebi.ac.uk/Tools/clustalw2/index.html) or “BLAST 2 Sequences” using default parameters (Tatusova, T. et al., FEMS Microbiol. Lett., 174:187-188 (1999)).

Active variants of exotoxins or exotoxin fragments can be prepared using suitable methods, for example, by direct synthesis, mutagenesis (e.g., site directed mutagenesis, scanning mutagenesis) and other methods of recombinant DNA technology. Active variants can be identified and/or selected using a suitable cytotoxicity assay.

Fusion proteins comprising an exotoxin or a fragment of an exotoxin are also contemplated. A fusion protein may encompass a polypeptide comprising an exotoxin (e.g., SEQ ID NO:2, SEQ ID NO:4, or SEQ ID NO:6), an exotoxin fragment, or an active variant thereof as a first moiety, linked via a covalent bond (e.g., a peptide bond) to a second moiety (a fusion partner) not occurring in an exotoxin as found in nature. Thus, the second moiety can be an amino acid, oligopeptide or polypeptide. The second moiety can be linked to the first moiety at a suitable position, for example, the N-terminus, the C-terminus or internally. In one embodiment, the fusion protein comprises an affinity ligand (e.g., an enzyme, an antigen, an epitope tag, an antibody or antigen-binding fragment of an antibody, a binding domain) and a linker sequence as the second moiety, and an exotoxin or an exotoxin fragment as the first moiety. Additional (e.g., third, fourth) moieties can be present as appropriate. The second (and additional moieties) can be any amino acid, oligopeptide or polypeptide that does not interfere with the cytotoxic activity of the exotoxin. Fusion proteins can be prepared using suitable methods, for example, by direct synthesis, recombinant DNA technology, etc.

In certain embodiment, the fusion protein comprises a first moiety which shares at least about 85% sequence similarity or identity with an exotoxin (e.g., SEQ ID NO:2, SEQ ID NO:4, or SEQ ID NO: 6) or a fragment of an exotoxin, preferably at least about 90% sequence similarity or identity, and more preferably at least about 95%, at least about 96%, at least about 97%, at least about 98%, or at least about 99% sequence similarity or identity with the exotoxin or exotoxin fragment. Preferably, the percentage of identity is calculated over the full length of the first moiety.

The invention also relates to nucleic acids encoding a Pseudomonas aeruginosa exotoxin, a fragment thereof, or a variant thereof. Exemplary nucleotide sequences encoding ExoU, ExoT, and ExoU are disclosed herein. In certain embodiment, the nucleic acid comprises a sequence that is at least about 85% sequence similarity or identity with an exotoxin-coding sequence (e.g., SEQ ID NO:1, SEQ ID NO:3, or SEQ ID NO: 5) or a fragment of an exotoxin, preferably at least about 90% sequence similarity or identity, and more preferably at least about 95%, at least about 96%, at least about 97%, at least about 98%, or at least about 99% sequence similarity or identity with the sequence that encodes an exotoxin or exotoxin fragment.

4. Nucleic Acid Constructs Emcpdomg P. Aeruginosa Exotoxins

The invention provides recombinant nucleic acids that encode P. aeruginosa exotoxins and cytotoxic fragments thereof. The sequence encoding the exotoxin or cytotoxic fragment is operably liked to a suitable control element, such that the exotoxin or cytotoxic fragment is produced in a cancer cell. The recombinant nucleic acid can be in any desired form, such as a non-viral vector, a plasmid, RNA, DNA, and the like. In preferred aspects, the recombinant nucleic acid is a recombinant viral vector. The recombinant viral vector can be an isolated nucleic acid molecule, or a nucleic acid molecule that is associated with a suitable delivery system, or in the form of a recombinant virus.

A. Recombinant Viruses and Viral Vectors

In certain embodiments, the nucleic acid encoding the exotoxin or exotoxin fragment of the invention is inserted into a recombinant viral vector. In certain embodiments, the recombinant viral vector is a Vaccinia virus vector, an Adenovirus vector, an Herpes simplex virus vector, a Newcastle Disease Virus vector, a Reovirus vector, a Coxsackievirus vector, or Senneca Valley Virus vector. Other suitable viral vectors include, e.g., an adeno-associated virus vector, a lentivirus vector, or an alphavirus vector.

In certain embodiments, replication-deficient viral vectors are preferred.

Vaccinia virus (VV) is a member of the genus Orthopoxvirus of the family Poxviridae. Several unique features of VV make it an excellent choice as a gene delivery vehicle in vivo. First, VV has a wide host range, capable of infecting almost all human cell types with high efficiency. Many other viruses have a more restricted host cell range. For example, infection by adenovirus is dependent on the abundance of the cell surface receptor coxsackievirus and adenovirus receptor (CAR), so cells that lack CAR are usually poorly infected by adenovirus. Second, VV infection and gene expression occur extremely efficiently. A number of viral promoters can be chosen from to control the timing and level of transgene expression. Third, the VV genome can accommodate at least 25 kb of foreign DNA sequence. This quantity could be further expanded by deleting viral DNA that is not required for replication in cultured cells. In comparison, other commonly used vector systems, such as adenovirus, adeno-associated virus, and retrovirus, can accommodate considerably less foreign DNA. Last, VV replication occurs exclusively in the cytoplasm, eliminating the possibility of chromosomal integration, in contrast to the retrovirus delivery system.

Several highly attenuated VV strains have been employed as the vectors to enhance the safety of VV vectors. For example, modified vaccinia virus Ankara (MVA) was obtained from serial passages in cultures of chicken embryo fibroblasts, resulting in the loss of substantial genomic information, including many genes regulating virus-host interactions. The virus has lost the ability to replicate in mammalian cells and became apathogenic even for immunodeficient animals. Importantly, the ability of MVA to infect and synthesize viral proteins (and transgene products) is not impaired. Similarly, NYVAC is a derivative of the Copenhagen strain with multiple deletions whose replication in human cells is markedly impaired. Attenuated VV strain Lister and its derivatives have been evaluated in a number of studies as well. A recombinant VV that expressed the tumor suppressor p53 gene has also been created. The virus (rVV-p53) was built on the attenuated Lister strain backbone.

The genomes of several of the vaccinia strains have been completely sequenced. Many of the essential and nonessential genes are identified. Due to high sequence homology among different strains, genomic information from one vaccinia strain can be used for designing and generating recombinant viruses in other strains. And most importantly, the techniques for production of recombinant vaccinia strains by genetic engineering have been well documented (Moss, Curr. Opin. Genet. Dev. 3 (1993), 86-90; Broder and Earl, Mol. Biotechnol. 13 (1999), 223-245; Timiryasova et al., Biotechniques 31 (2001), 534-540).

Many loci in the vaccinia genome have been used as the integration sites of foreign genes. For example, the non-essential TK gene is frequently used as the insertion site. Other frequently used non-essential regions include the HA gene. It has been shown that, even though the same expression cassettes are used, significant variation of protein expression can occur dependent on the location of insertion sites (Coupar et al., J. Gen. Virol. 81 (2000), 431-439).

The non-essential regions of the vaccinia genome, e.g. the tk gene, can be used for the insertion of foreign genes without significantly affecting viral replication and infection. See, e.g., Shen et al., Fighting Cancer with Vaccinia Virus: Teaching New Tricks to an Old Dog, Molecular Therapy (2005) 11, 180-195; doi: 10.1016/j.ymthe.2004.10.015.

The adenovirus genome is a linear double-stranded DNA molecule of approximately 36,000 base pairs with the 55-kDa terminal protein covalently bound to the 5′ terminus of each strand. Adenoviral (“Ad”) DNA contains identical Inverted Terminal Repeats (“ITRs”) of about 100 base pairs with the exact length depending on the serotype. The viral origins of replication are located within the ITRs exactly at the genome ends.

Unlike retroviruses, which integrate into the host genome, adenoviruses persist extrachromosomally thus minimizing the risks associated with insertional mutagenesis (Haj-Ahmad and Graham (1986) J. Virol. 57:267-74; Bett et al. (1993) J. Virol. 67:5911-21; Mittereder et al. (1994) Human Gene Therapy 5:717-29; Seth et al. (1994) J. Virol. 68:933-40; Barr et al. (1994) Gene Therapy 1:51-58; Berkner, K. L. (1988) BioTechniques 6:616-29; and Rich et al. (1993) Human Gene Therapy 4:461-76).

Use of Adenovirus-derived vectors for gene therapy is known in the art. See, for example, U.S. Pat. No. 6,908,762, U.S. Pat. No. 6,756,226, U.S. Pat. No. 5,824,544; U.S. Pat. No. 5,707,618; U.S. Pat. No. 5,693,509; U.S. Pat. No. 5,670,488; U.S. Pat. No. 5,585,362.

Adenoviral vectors for use with the present invention may be derived from any of the various adenoviral serotypes, including, without limitation, any of the over 40 serotype strains of adenovirus, such as serotypes 2, 5, 12, 40, and 41.

Herpes simplex virus (HSV) has been suggested to be of use both as a delivery vector, and for the oncolytic treatment of cancer. For the treatment of cancer, which may also include the delivery of gene(s) enhancing the therapeutic effect, a number of mutations to HSV have been identified which still allow the virus to replicate in culture or in actively dividing cells in vivo (e.g. in tumors), but which prevent significant replication in normal tissue. Such mutations include disruption of the genes encoding ICP34.5, ICP6 and thymidine kinase. Of these, viruses with mutations to ICP34.5, or ICP34.5 together with mutations of e.g. ICP6 have so far shown the most favorable safety profile. Viruses deleted for only ICP34.5 have been shown to replicate in many tumor cell types in vitro and to selectively replicate in artificially induced brain tumors in mice while sparing surrounding tissue. Early stage clinical trials have also shown their safety in human.

Herpes simplex virus type 1 (HSV-1) is the most extensively engineered herpesvirus for purposes of gene transfer. HSV has a large genome composed of 152 kb of linear dsDNA containing at least 84 almost entirely contiguous (unspliced) genes, approximately half of which are nonessential for virus replication in cell culture. These features provide for multiple sites of foreign gene insertion, making HSV a large capacity vector capable of harboring at least 30 kb of non-HSV sequences representing large single genes or multiple transgenes that may be coordinately or simultaneously expressed. Highly defective mutants deleted for the five immediate early (IE) genes do not express the remaining lytic viral functions and are essentially silent except for transgene expression. These vectors can be grown to high titer in complementing cell lines without the production of detectable replication competent virus. The IE gene deletion vectors are non-cytotoxic yet are capable of persisting in a state similar to latency in neurons and other cell types within non-neuronal tissue. A most attractive feature is the efficient infectivity of HSV for a large number of cell types, which results in efficient gene transduction. Efficient infectivity and transduction has made possible repeat vector administration even in immune hosts. Limitations of these vectors include the lack of experience with recombinant herpesviruses in patients, difficulties related to long-term transgene expression in certain tissues including brain and difficulties related to vector targeting, since the mechanism of HSV attachment and entry is complex, involving multiple viral envelope glycoproteins.

HSV amplicon vectors represent an alternative to replication defective, recombinant genomic vectors. Amplicon plasmids are based on defective interfering virus genomes that arise on high passage of virus stocks. They are generally approximately 15 kb in length and minimally possess a viral origin of replication and packaging sequences. The standard amplicon system requires the functions of helper HSV for particle production and packaging of genome length concatemerized vector DNA. Amplicon vector production has been improved through use of helper virus genome plasmids deleted for packaging signals; the helper genomes are propagated in bacteria as bacterial artificial chromosomes. These preparations are advantaged by being nearly helper-free; however, until the helper DNA is completely devoid of sequences shared with the amplicon vector (for example, origin of replication), recombination between the amplicon and helper DNA will occur raising the possibility of contamination of vector stocks with unwanted recombinants, some of which may be replication competent. Production systems dependent on transfection are also difficult to scale-up, and have not yet produced high titer vector. Thus far, production of replication-competent virus-free genomic vectors using complementing cells results in a 2-3 log higher vector particle yield using a less complicated production system.

Newcastle disease virus (NDV) is a member of the Avulavirus genus of the Paramyxoviridae family (Fauquet et al., 2005. Virus Taxonomy: Eighth Report of the International Committee on Taxonomy of Viruses, Academic Press). There are several advantages of using NDV as a vector for mammals (Bukreyev et al., 2006. J Virol 80: 10293-306). One of the advantages results from the fact that mammals are not natural hosts for NDV. Despite the restricted replication of NDV in mammals, foreign genes can be expressed efficiently from the NDV genome and several promising NDV-based vector vaccines for use in mammals have been developed already (Bukreyev and Collins, 2008. Curr Opin Mol Ther 10: 46-55; Bukreyev et al., 2005. J Virol 79: 13275-84; DiNapoli et al., 2007. Proc Natl Acad Sci USA 104: 9788-93; Dinapoli, et al., 2009. Vaccine 27: 1530-9; DiNapoli et al., 2007. J Virol 81: 11560-8).

Adeno Associated Virus (AAV) is a parvovirus which belongs to the genus Dependovirus. AAV has several attractive features not found in other viruses. First, AAV can infect a wide range of host cells, including non-dividing cells. Second, AAV can infect cells from different species. Third, AAV has not been associated with any human or animal disease and does not appear to alter the biological properties of the host cell upon integration. Indeed, it is estimated that 80-85% of the human population has been exposed to the virus. Finally, AAV is stable at a wide range of physical and chemical conditions, facilitating production, storage and transportation.

The AAV genome is a linear single-stranded DNA molecule containing approximately 4681 nucleotides. The AAV genome generally comprises an internal non-repeating genome flanked on each end by inverted terminal repeats (ITRs). The ITRs are approximately 145 base pairs (bp) in length. The ITRs have multiple functions, including serving as origins of DNA replication and as packaging signals for the viral genome.

AAV is a helper-dependent virus; that is, it requires co-infection with a helper virus (e.g., adenovirus, herpesvirus or vaccinia) in order to form AAV virions in the wild. In the absence of co-infection with a helper virus, AAV establishes a latent state in which the viral genome inserts into a host cell chromosome, but infectious virions are not produced. Subsequent infection by a helper virus rescues the integrated genome, allowing it to replicate and package its genome into infectious AAV virions. While AAV can infect cells from different species, the helper virus must be of the same species as the host cell. Thus, for example, human AAV will replicate in canine cells co-infected with a canine adenovirus.

Use of AAV-derived vectors for gene therapy is known in the art. See, for example, U.S. Pat. No. 6,489,162, U.S. Pat. No. 5,474,935; U.S. Pat. No. 5,139,941; U.S. Pat. No. 5,622,856; U.S. Pat. No. 5,658,776; U.S. Pat. No. 5,773,289; U.S. Pat. No. 5,789,390; U.S. Pat. No. 5,834,441; U.S. Pat. No. 5,863,541; U.S. Pat. No. 5,851,521; U.S. Pat. No. 5,252,479.

Retroviruses also provide a convenient platform for gene delivery. A selected gene can be inserted into a vector and packaged in retroviral particles using techniques known in the art. The recombinant virus can then be isolated and delivered to cells of the subject either in vivo or ex vivo. A number of retroviral systems have been described. See, e.g., U.S. Pat. No. 5,219,740; Miller and Rosman (1989) BioTechniques 7:980-90; Miller, A. D. (1990) Human Gene Therapy 1:5-14; Scarpa et al. (1991) Virology 180:849-52; Burns et al. (1993) Proc. Natl. Acad. Sci. USA 90:8033-37; Boris-Lawrie and Temin (1993) Curr. Opin. Genet. Develop. 3:102-09.

Replication-defective murine retroviral vectors are widely used gene transfer vectors. Murine leukemia retroviruses include a single stranded RNA molecule complexed with a nuclear core protein and polymerase (pol) enzymes, encased by a protein core (gag), and surrounded by a glycoprotein envelope (env) that determines host range. The genomic structure of retroviruses includes gag, pol, and env genes and 5′ and 3′ long terminal repeats (LTRs). Retroviral vector systems exploit the fact that a minimal vector containing the 5′ and 3′ LTRs and the packaging signal are sufficient to allow vector packaging, infection and integration into target cells, provided that the viral structural proteins are supplied in trans in the packaging cell line. Fundamental advantages of retroviral vectors for gene transfer include efficient infection and gene expression in most cell types, precise single copy vector integration into target cell chromosomal DNA and ease of manipulation of the retroviral genome.

Lentivirus is a genus of slow viruses of the Retroviridae family, characterized by a long incubation period. Lentiviruses can deliver a significant amount of genetic information into the DNA of the host cell and have the unique ability among retroviruses of being able to replicate in non-dividing cells, so they are one of the most efficient methods of a gene delivery vector. HIV, SW, and FIV are all examples of lentiviruses. Use of lentiviral-derived vectors for gene therapy is known in the art. See, for example, U.S. Pat. No. 6,800,281, U.S. Pat. No. 6,277,633.

Additional viral vectors useful for delivering the nucleic acid molecules include those derived from the pox family of viruses, including avian poxvirus.

Avipoxviruses, such as the fowlpox and canarypox viruses, can be used to deliver the genes. Recombinant avipox viruses expressing immunogens from mammalian pathogens are known to confer protective immunity when administered to non-avian species. The use of avipox vectors in human and other mammalian species is advantageous with regard to safety because members of the avipox genus can only productively replicate in susceptible avian species. Methods for producing recombinant avipoxviruses are known in the art and employ genetic recombination, as described above with respect to the production of vaccinia viruses. See, e.g., WO 91/12882; WO 89/03429; and WO 92/03545.

The nucleic acid molecule may also be delivered using an alphavirus-derived vector. Many properties of alphavirus vectors make them a desirable alternative to other virus-derived nucleic acid delivery systems being developed, including the ability to (i) rapidly engineer expression constructs, (ii) produce high-titered stocks of infectious particles, (iii) infect non-dividing cells, and (iv) attain high levels of expression (Strauss and Strauss, Microbiol. Rev. 1994, 58:491-562; Liljestrom et al., Biotechnology 1991, 9:1356-1361; Bredenbeek et al., Semin. Virol. 1992, 3:297-310; Xiong et al., Science 1993, 243:1188-1191). Defective Sindbis viral vectors have been used to protect mammals from protozoan parasites, helminth parasites, ectoparasites, fungi, bacteria, and viruses (PCT Publication No. WO 94/17813).

B. Non-Viral Vectors

In certain embodiments, the nucleic acid encoding the exotoxin or exotoxin fragment of the invention is inserted into a non-viral vector. A non-viral vector is typically an autonomously replicating, extrachromosomal nucleic acid molecule that is distinct from the genome of the host cell, and is not assembled into a viral particle or capsid by a host cell. Sometimes, non-viral vectors may offer certain advantages over a recombinant viral vector, such as: ease in the preparation and modification; greater flexibility with respect to the size of the genetic material to transfect; greater safety in vivo; and diminished immune response. The non-viral vectors described herein generally comprise a sequence that encode a P. aeruginosa exotoxin or a cytotoxic fragment thereof that is operably linked to one or more control elements, such that the exotoxin or cytotoxic fragment is produced in a cancer cell.

The invention also relates to methods of making a recombinant vector, such as a recombinant viral vector, for delivering an exotoxin or exotoxin fragment described herein to a host cell, the method comprising: (i) providing a recombinant vector, such as a viral vector, (ii) inserting a nucleic acid sequence encoding said Pseudomonas aeruginosa exotoxin into said viral vector, wherein the exotoxin-coding seqeunce is operably linked to an expression control sequence, such that said exotoxin is produced in said host cell.

The nucleic acid molecule described herein generally comprises a nucleotide sequence that encodes an exotoxin or exotoxin fragment that is operably linked to an expression control sequence that controls the expression of the exotoxin or exotoxin fragment in a mammalian cell. Suitable expression control elements such as promoters, enhancers, ribosome entry sites, polyadenylation sequences, and/or IRES, and the like, are well known in the art.

For example, a suitable promoter may be used to control the expression of an exotoxin or exotoxin fragment. Other expression control sequences contemplated for use in the invention include enhancers, introns, polyadenylation signal, and 3′UTR sequences.

5. Delivery of Pseudomonas Aeruginosa Cytotoxins

Modes of delivery of an exotoxin or exotoxin fragment include direct delivery of the protein/peptide, and/or administering a nucleic acid molecule encoding the exotoxin or exotoxin fragment. Preferably, Pseudomonas exotoxin is preferentially delivered to tumor cells, thereby optimizing tumor killing and minimizing potential toxicity.

In preferred embodiments, the exotoxin or exotoxin fragment of the invention is provided by a nucleic acid molecule encoding the exotoxin or exotoxin fragment. The nucleic acid molecule may be a DNA molecule, an RNA molecule, or may contain a DNA portion, or an RNA portion. Preferably, the nucleic acid is a recombinant viral vector comprising a sequence encoding an exotoxin or exotoxin fragment described herein. Examples of recombinant viral vectors include, e.g., vectors derived from Vaccinia virus, Newcastle Disease Virus, Reovirus, Coxsackievirus, Senneca Valley Virus, retrovirus, adenovirus, herpes virus, pox virus, or adeno-associated virus (AAV). The nucleic acid can be delivered as a non-viral vector.

Recombinant viral vector can be delivered to a host cell in the form of a recombinant virus. Recombinant viral vector can be packaged into viral coats or capsids by any suitable procedure, for example, by transfecting the recombinant viral vector into a packaging cell line. Any suitable packaging cell line can be used to generate recombinant virus. Suitable packaging lines for retroviruses include derivatives of PA317 cells, ψ-2 cells, CRE cells, CRIP cells, E-86-GP cells, and 293GP cells. Line 293 cells can be used for adenoviruses and adeno-associated viruses. Neuroblastoma cells can be used for herpes simplex virus, e.g. herpes simplex virus type 1. Sometimes, a helper virus (which provides missing proteins for production of new virions) may be needed to produce a recombinant virus described herein.

Nucleic acid molecules encoding an exotoxin or exotoxin fragment can also be delivered using a non-viral based nucleic acid delivery system. For example, methods of delivering a nucleic acid to a target cell have been described in U.S. Pat. Nos. 6,413,942, 6,214,804, 5,580,859, 5,589,466, 5,763,270 and 5,693,622.

Nucleic acid molecules described herein can be packaged in liposomes prior to delivery to a subject or to cells, as described in U.S. Pat. Nos. 5,580,859, 5,549,127, 5,264,618, 5,703,055. For a review of the use of liposomes as carriers for delivery of nucleic acids, see, Hug and Sleight (1991) Biochim. Biophys. Acta. 1097:1-17; Straubinger et al. (1983) in Methods of Enzymology Vol. 101, pp. 512-27; de Lima et al. (2003) Current Medicinal Chemistry, Volume 10(14): 1221-31. Representative liposomes include, but not limited to cationic liposomes, optionally coated with polyethylene glycol (PEG) to reduce non-specific binding of serum proteins and to prolong circulation time. See Koning et al., 1999; Nam et al., 1999; and Kirpotin et al., 1997. Temperature-sensitive liposomes can also be used, for example THERMOSOMES™ as disclosed in U.S. Pat. No. 6,200,598. The use of vector-liposome complexes has been described in U.S. Pat. No. 5,851,818. Liposomes can be prepared by any of a variety of techniques that are known in the art. See e.g., Betageri et al., 1993; Gregoriadis, 1993; Janoff, 1999; Lasic & Martin, 1995; Nabel, 1997; and U.S. Pat. Nos. 4,235,871; 4,551,482; 6,197,333; and 6,132,766.

Nucleic acids can also be delivered in cochleate lipid compositions similar to those described by Papahadjopoulos et al. (1975) Biochem. Biophys. Acta. 394:483-491. See also U.S. Pat. Nos. 4,663,161 and 4,871,488. For example, a plasmid vector may be complexed with Lipofectamine 2000. Wang et al. (2005) Mol. Therapy 12(2):314-320.

Biolistic delivery systems employing particulate carriers such as gold and tungsten may also be used to deliver nucleic acids (e.g., recombinant viral vectors and non-viral vectors). The particles are coated with the vector and accelerated to high velocity, generally under reduced pressure, using a gun powder discharge from a “gene gun.” See, e.g., U.S. Pat. Nos. 4,945,050, 5,036,006, 5,100,792, 5,179,022, 5,371,015, and 5,478,744.

A wide variety of other methods can be used to deliver the nucleic acids described herein. Such methods include DEAE dextran-mediated transfection, calcium phosphate precipitation, polylysine- or polyornithine-mediated transfection, or precipitation using other insoluble inorganic salts, such as strontium phosphate, aluminum silicates including bentonite and kaolin, chromic oxide, magnesium silicate, talc, and the like. Other useful methods of transfection include electroporation, sonoporation, protoplast fusion, peptoid delivery, or microinjection. See, e.g., Sambrook et al (1989) Molecular Cloning, A Laboratory Manual, Cold Spring Harbor Laboratories, New York, for a discussion of techniques for transforming cells of interest; and Felgner, P. L. (1990) Advanced Drug Delivery Reviews 5:163-87, for a review of delivery systems useful for gene transfer. Exemplary methods of delivering DNA using electroporation are described in U.S. Pat. Nos. 6,132,419; 6,451,002, 6,418,341, 6,233,483, U.S. Patent Publication No. 2002/0146831, and International Publication No. WO 00/45823.

The exotoxins and exotoxin fragments described herein may also be delivered as protein therapeutics. Methods for direct delivery of the peptides/proteins are known in the art. For example, a polymer-based intracellular delivery system may be used. See, e.g., U.S. Application Publication No. 2004/0101941, which describes intracellular delivery of a protein by conjugating the protein to a polymer such as an N-alkyl acrylamide polymer. Alternatively, cationic lipids can be used for intracellular delivery of a protein (e.g., by encapsulating the protein in a cationic liposome, or associating the protein to form a lipoplex; see e.g., U.S. Application Publication No. 20030008813).

Additionally, an exotoxin or exotoxin fragment described herein can be delivery as a fusion protein. The fusion protein may comprise an exotoxin or exotoxin fragment, and a fusion partner that preferentially target cancer cells. For example, a fusion protein comprising (i) an exotoxin or exotoxin fragment and (ii) an antibody (or an antigen-binding fragment thereof) that recognize a tumor-specific antigen can be used. Alternatively, the fusion parter can be protein A subunit of AB toxins, or Azurin p18 (which selectively target transformed cancer cells in vivo), or a ligand that can be recognized by a cell surface receptor of a cancer cell.

Bacteria have been used to deliver various toxins directly into solid tumors in vivo. See, e.g., Lemmon, M. J., et al., Anaerobic bacteria as a gene delivery system that is controlled by the tumor microenvironment. Gene Ther, 1997. 4(8): p. 791-6. Fox, M. E., et al., Anaerobic bacteria as a delivery system for cancer gene therapy: in vitro activation of 5-fluorocytosine by genetically engineered clostridia. Gene Ther, 1996. 3(2): p. 173-8. A potential concern for bacterium-based devliery is the possibility of infection.

6. Pharmaceutical Compositions and Methods of Administration

In another aspect, the invention relates to an exotoxin or exotoxin fragment as described herein for use in therapy (e.g., for treating cancer).

The invention provides a pharmaceutical composition comprising a recombinant virus, wherein said virus comprises a recombinant viral vector encoding an exotoxin, or a fragment of an exotoxin, as described herein.

The invention also provides a pharmaceutical composition comprising a nucleic acid molecule comprising a nucleotide sequence that encodes an exotoxin, or a fragment of an exotoxin, as described herein. In certain embodiment, the nucleic acid molecule is a recombinant viral vector. In certain embodiment, the nucleic acid molecule is a non-viral vector. Optionally, a delivery system for delivering the nucleic acid (e.g., liposomes) may be provided. The delivery system can be co-formulated with the nucleic acid into a pharmaceutical composition, or supplied separately (e.g., in a separate container in a kit). If provided separately, the delivery system may be mixed with the nucleic acid prior to administration.

The pharmaceutical composition may further comprise one or more pharmaceutically acceptable carriers, diluents, or excipients. Such excipients include any pharmaceutical agent that does not itself induce the production of antibodies harmful to the individual receiving the composition, and which may be administered without undue toxicity. Pharmaceutically acceptable excipients include, but are not limited to, sorbitol, Poloxamer (Pluronic F68), any of the various TWEEN compounds, and liquids such as water, saline, glycerol and ethanol. Pharmaceutically acceptable salts can be included therein, for example, mineral acid salts such as hydrochlorides, hydrobromides, phosphates, sulfates, and the like; and the salts of organic acids such as acetates, propionates, malonates, benzoates, and the like. Additionally, auxiliary substances, such as wetting or emulsifying agents, pH buffering substances, and the like, may be present in such vehicles. A thorough discussion of pharmaceutically acceptable excipients is available in Remington's Pharmaceutical Sciences (Mack Pub. Co., N.J. 1991).

One particularly useful formulation comprises the nucleic acid (e.g., a viral or non-viral vector) in combination with one or more dihydric or polyhydric alcohols, and, optionally, a detergent, such as a sorbitan ester. See, e.g., International Publication No. WO 00/32233.

In certain embodiments, the nucleic acid molecules as described herein are administered to a mammalian subject with cancer to treat the cancer. In certain embodiments, a protein comprising an exotoxin or exotoxin fragment as described herein are administered to a mammalian subject with cancer to treat the cancer. In certain embodiments, the mammalian subject is a human. For example, the subject may be suffering from or susceptible to cancer. Treatment a disease (such as cancer) includes preventing or delaying the onset or progression of the diseases, mitigating the severity of the diseases, or protecting the cells from further damages, or ameliorating symptoms. Treatment also includes prophylactic treatment of a subject that has not manifested a disease phenotype.

In certain embodiments, the cancer patient may be suffering from or susceptible to: cancer of the bladder, breast, colon, kidney, liver, lung (including small cell lung cancer), esophagus, gall bladder, ovary, pancreas, stomach, cervix, thyroid, prostate, testicle, and skin, including (squamous cell carcinoma); leukemia, acute lymphocytic leukemia, acute lymphoblastic leukemia, B-cell lymphoma, T-cell lymphoma, Hodgkin's lymphoma, non-Hodgkin's lymphoma, hairy cell lymphoma and Burkett's lymphoma; acute and chronic myelogenous leukemia, myelodysplastic syndrome and promyelocytic leukemia; fibrosarcoma, rhabdomyosarcoma; astrocytoma, neuroblastoma, glioma and schwannomas; melanoma, seminoma, teratocarcinoma, osteosarcoma, xenoderoma pigmentosum, keratoctanthoma, thyroid follicular cancer and Kaposi's sarcoma.

An effective amount is administered to a subject in need thereof. An “effective amount” or “therapeutically effective amount” is an amount that is sufficient to achieve the desired therapeutic or prophylactic effect under the conditions of administration, such as an amount sufficient to reduce/ameliorate symptoms of cancer, to reduce the proliferation of cancer cells, etc.

One of skill in the art can determine an effective dose empirically. Methods of determining the most effective means and dosages of administration are well known to those of skill in the art and will vary with the pharmaceutical composition, the target cells, and the subject being treated. The dosage administered, as single or multiple doses, to an individual will vary depending upon a variety of factors, including pharmacokinetic properties, the route of administration, patient conditions and characteristics (sex, age, body weight, health, size), extent of symptoms, concurrent treatments, frequency of treatment and the effect desired. Therapeutically effective doses can be readily determined by one of skill in the art and will depend on the particular delivery system used. Administration can be effected in one dose, continuously or intermittently throughout the course of treatment. Single and multiple administrations can be carried out with the suitable dose level and pattern being selected by the clinician.

If a nucleic acid molecule (such as a recombinant viral vector) encoding the exotoxin or exotoxin fragment is administered, an effective amount of a pharmaceutical formulation will usually deliver a dose of from about 10 ng to about 1 g nucleic acid per patient. Doses for viral vectors generally vary from about 1 to about 10000 virions per dose.

If the exotoxin or exotoxin fragment is administered directly as a protein, an effective amount of a pharmaceutical formulation will generally deliver a dose of about 0.001 to about 100 mg/kg body weight.

In a multiple dose schedule the various doses may be given by the same or different routes. Multiple doses can be administered at any desired interval, such as at least 1 week apart (e.g., about 2 weeks, about 3 weeks, about 4 weeks).

If desired, a second therapeutic agent may be used to in combination with the exotoxins or exotoxin fragments described herein. Examples of such second therapeutic agents include, e.g., 5-fluorouracil, mitomycin C, methotrexate, hydroxyurea, cyclophosphamide, dacarbazine, mitoxantrone, anthracyclins, carboplatin, cisplatin, taxol, taxotere, tamoxifen, anti-estrogens, and interferons.

Typically, therapeutic proteins and nucleic acids are administered parenterally, intravenously (iv), intramuscularly (im), intraperitoneally (ip) or subcutaneously (sc), although other routes of administration are also possible, e.g., orally or intranasally.

### EXEMPLIFICATION

The invention now being generally described, it will be more readily understood by reference to the following examples, which are included merely for purposes of illustration of certain aspects and embodiments of the present invention, and are not intended to limit the invention.

Introduction

Cancer remains amongst the deadliest diseases world-wide [1]. For example just for breast cancer, in the United States alone, 207,090 new cases and 39,840 deaths occur annually [2]. Treatments for most patients often include a combination of surgery, chemotherapy, hormone therapy and ionizing radiation. In general, these treatments are at least partially effective at the beginning, however, after a variable period of time, progression occurs. At that point, resistance to therapy is not only common but expected [3]. Innovative therapies are needed to overcome these problems and successfully treat primary breast tumors and prevent breast tumor metastases.

There are several reasons explaining the failures of current therapies.

(1) Therapy has limited cellular targets. One of the major mechanisms by which tumor cells become resistant to chemotherapy is by inserting mutations in the cellular targets of chemotherapy drugs [4, 5]. Development of cancer resistance to therapy by this mechanism is achieved more easily if the therapy has limited cellular targets. Thus, the fewer cellular targets a drug has, the easier it is for cancer resistance to develop by this mechanism. For example, diphtheria toxin (DT) and ExoA, two popular bacteria toxin-based cancer drugs [6-8], ADP-ribosylate eEF-2, which halts protein synthesis and induces cytotoxicity in tumor cells [9]. However, mutations in eEF-2, conferring resistance to these toxins, have already been reported [10].

(2) Therapy targets biosynthetic cellular processes for which physiological responses are present. Many breast cancer drugs induce cytotoxicity by inhibiting biosynthetic pathways, such as transcription, translation, DNA synthesis, and protein synthesis [3, 11]. Tumor cells can develop resistance to these chemotherapeutic agents by upregulating anti-apoptotic proteins such as BCL-2 or BCL-X, or by down-regulating death signaling and/or death proteins, such as p53 [12-14].

(3) Drug resistance pumps. Metabolic detoxification and drug expulsion from the cytoplasm by ATP-driven multidrug resistance (MDR) pumps is yet another important mechanism of resistance [12] by which small cancer drugs, such as cisplatin, campthothecin, and cyclophosphamide, may be eliminated.

(4) Therapy does not induce an effective anti-tumor immune response. The immune system is an important barrier to the growth of tumors and its recurrence [15-17]. Breast cancer is traditionally considered as a poorly immunogenic tumor [18, 19]. Various approaches, including the inclusion of immune-modulatory factors or immunogenic cytotoxins have been used to reduce the tumor-associated immuno-suppression and/or to promote the long-term antitumor immunity, thus overcoming low immunogenicity in cancer [19]. Effective destruction of tumors by the immune system likely depends on the number and functional abilities of tumor-specific CD8 T cells generated within tumors and has been reported to correlate with a positive prognosis for treatment [20].

(5) The induction of apoptotic compensatory proliferation signaling. Many anti-cancer drugs promote apoptosis in tumor cells [21-23]. However, it has become evident that resistance to apoptosis occurs frequently in cancer [24]. One possible reason for resistance to pro-apoptotic chemotherapy may be the existence of apoptotic compensatory proliferation signaling emitted by apoptotic/dying cells in response to therapy. For nearly three decades, it has been postulated that apoptotic cells induce proliferation in neighboring cells as a means to compensate for their own demise and to control homeostasis [25-29]. This form of apoptotic-induced proliferation is commonly referred to as apoptotic compensatory proliferation signaling [30-32]. The players and the mechanisms involved in compensatory proliferation signaling remain largely unknown. Apoptotic compensatory proliferation signaling could have far-reaching implication in cancer biology and in its treatment. As many current breast cancer drugs induce apoptosis in tumor cells [30-32], compensatory proliferation signaling could limit their effectiveness, contributing to the disappointing outcomes associated with these agents.

Therefore, Innovative therapies are needed to overcome these problems and successfully treat primary breast tumors and prevent breast tumor metastases.

Results and Discussion

Pseudomonas aeruginosa Exotoxins possess unique properties that make them candidates to be used alone or in combination to eradicate breast cancer metastases.

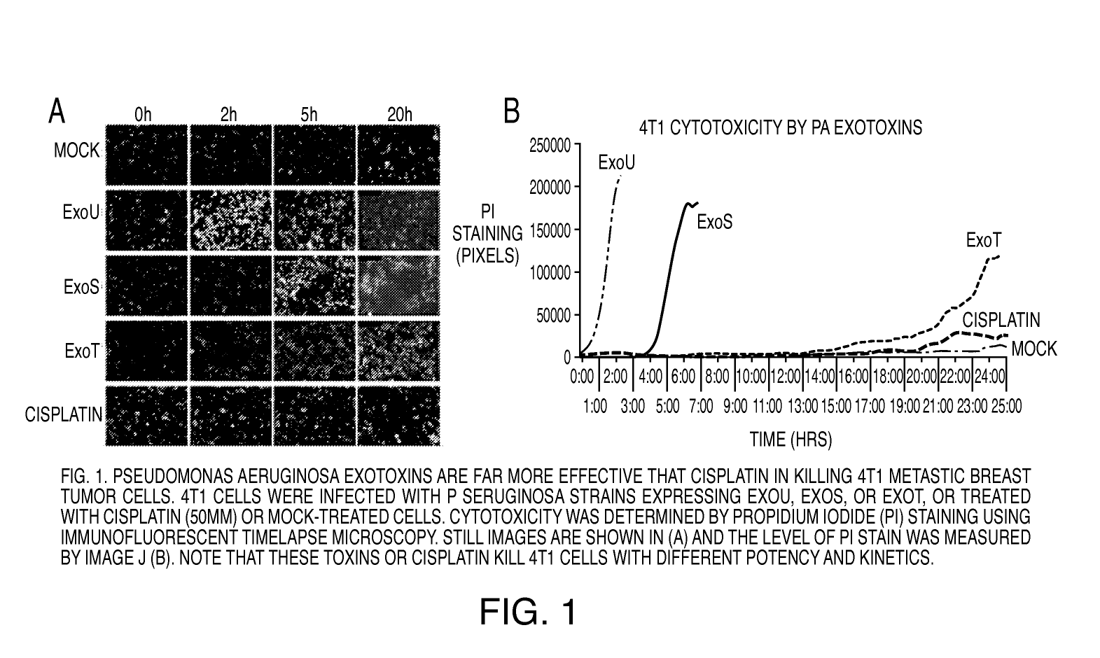

(i) Pseudomonas exotoxins are highly potent inducers of cell death. The results of our studies demonstrate that Pseudomonas exotoxins are far more potent inducers of cytotoxicity with faster kinetics of killing than standard chemotherapy in a variety of highly resistant and metastatic human and murine cancer cell lines, including 4T1, MCF-7, MDA-MB-231, AU-565, and EMT6 (breast cancer cell lines); B16 and A375 (melanoma cancer cell lines); Calu3, H1975, H1974, A549, and LLC (lung cancer cell lines), and HeLa cervical cancer cell line. For example, ExoU kills 100% of highly metastatic breast cancer cell line 4T1 within 2-3 hours, as indicated by propidium iodide (PI) staining (red) in FIG. 1. ExoU/S and T-mediated cell death is more effective than a known therapy, cisplatin, which we have found to induce cytotoxicity in approximately 30-50% of the same cancer cell line in 21+ hours. These toxins are also far more effective that the FDA approved bacterial toxin Exotoxin A (ExoA).

(ii) It is much more difficult for tumor cells to develop resistance to Pseudomonas Exotoxins because they target multiple cellular proteins and cellular processes that function in cell proliferation and survival [33, 34]. One of the major mechanisms by which tumor cells become resistant to chemotherapy is by inserting mutations in the cellular targets of chemotherapy drugs [4, 5]. In contrast, current standard chemotherapy drugs and bacteria toxin-based therapies, such as diphtheria toxin (DT), primarily affect one or limited cellular process [9, 35, 36]. For example, bacterial toxins DT and ExoA, two popular bacteria toxin-based cancer drugs [6-8], kill tumor cells by ADP-ribosylating eEF-2, thus halting protein synthesis and inducing cytotoxicity [9]. However, mutations in eEF-2, conferring resistance to these toxins, have already been reported [10].

(iii) Unlike current therapies that primarily induce apoptosis, a type of cytotoxicity which is generally believed to be anti-inflammatory in nature [37, 38], Pseudomonas Exotoxin-mediated cytotoxicities have been shown to induce a strong pro-inflammatory environment [39, 40]. The pro-inflammatory nature of these Exotoxins could lead to the generation of tumor-specific immune responses which may be critically important in eradicating tumor cells systemically. A successful therapy not only must be able to induce cytotoxicity in its target tumor cells, the killing must also culminate in an appropriate immune response against tumor [19].

(iv) The results of our studies demonstrate that Exotoxin T blocks a previously unknown pathway of tumor resistance that could be a major contributor to ineffectiveness of therapy in the treatment of breast cancer. It has long been postulated that dying cells induce compensatory proliferation in neighboring cells as a means to control homeostasis and maintain tissue integrity but the players and the mechanisms involved in the compensatory proliferation signaling have remained unknown [25, 41]. For the first time, we demonstrate that indeed the process of compensatory proliferation signaling does exist. As many current cancer drugs induce apoptosis in tumor cells [30-32], compensatory proliferation signaling could significantly limit their effectiveness. Importantly, our the results of our studies demonstrate that Exotoxin T both induces potent apoptosis and interferes with the ability of apoptotic HeLa cells to form compensatory complexes and induce proliferation in the surrounding cells. Our results indicate that apoptotic program cell death and the apoptotic compensatory proliferation signaling are distinct processes which can be uncoupled from each other in tumor cells following administration of ExoT, making this pathway highly attractive as a target for cancer therapy and Exotoxin T, a great candidate drug to accomplish this task.

(v) Exotoxin T is also a potent anti-proliferative agent. The results of our studies demonstrate that ExoT induces complete G1cell cycle arrest in all cancer cells that are resistant to its cytotoxicity, regardless of their origin, that we have examined thus. This feature can be quite useful in combination therapy.

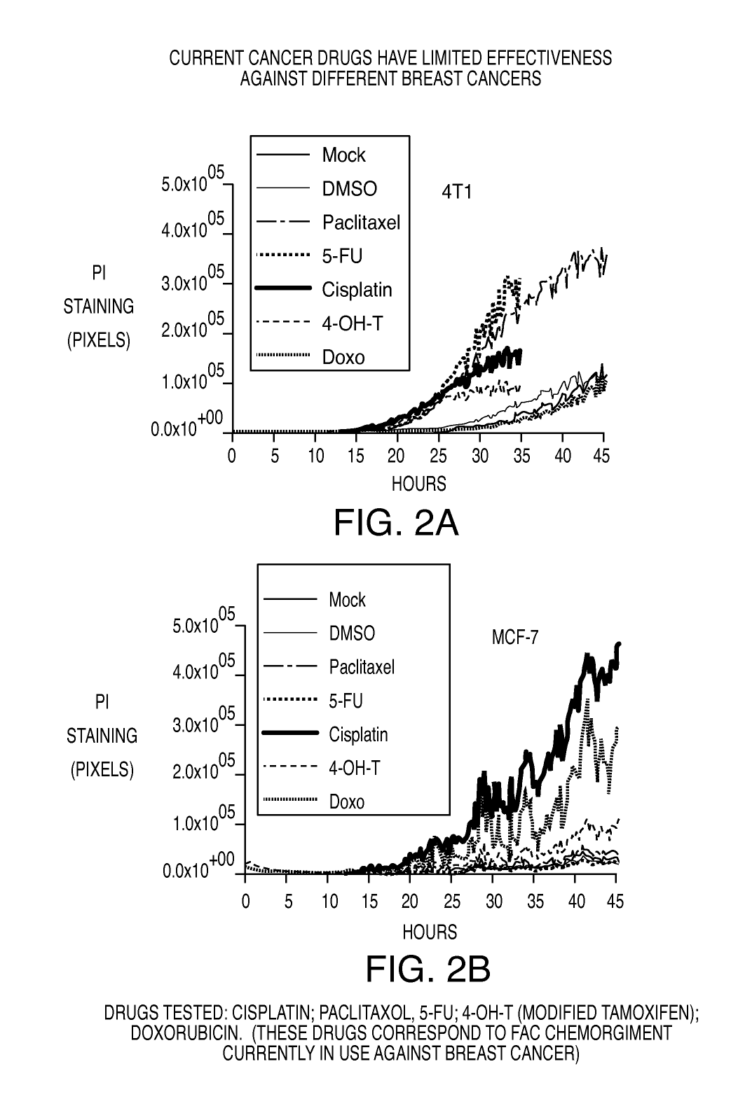

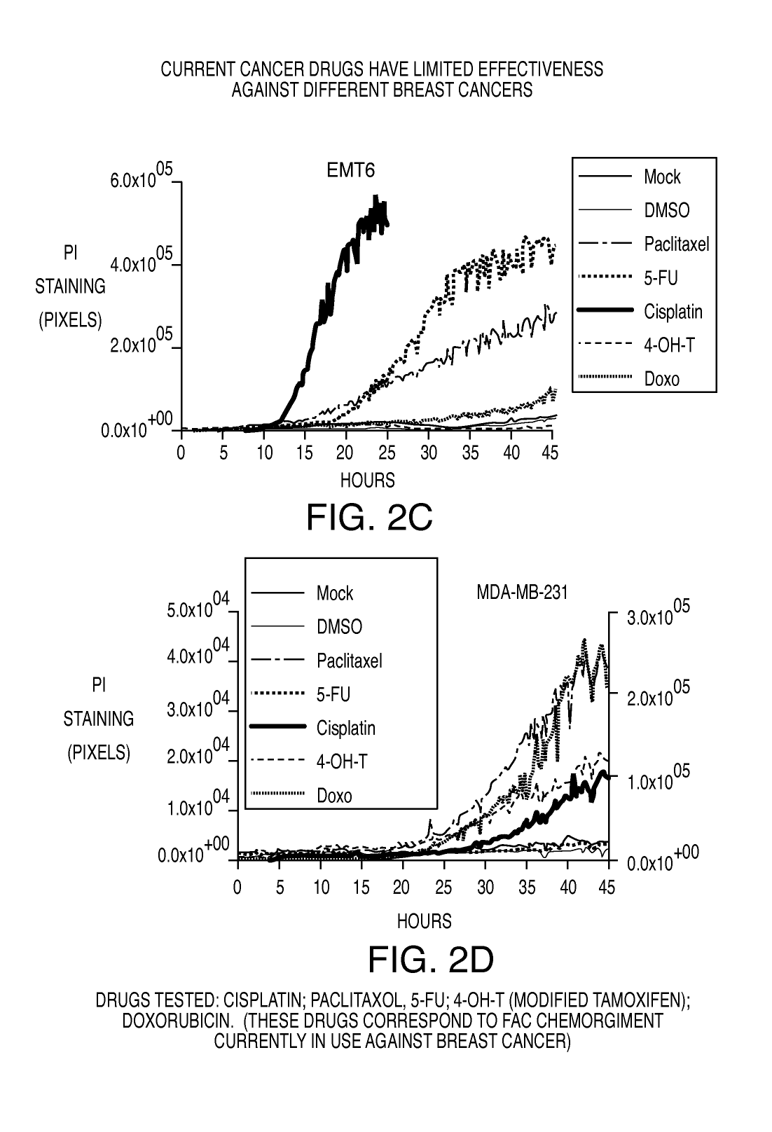

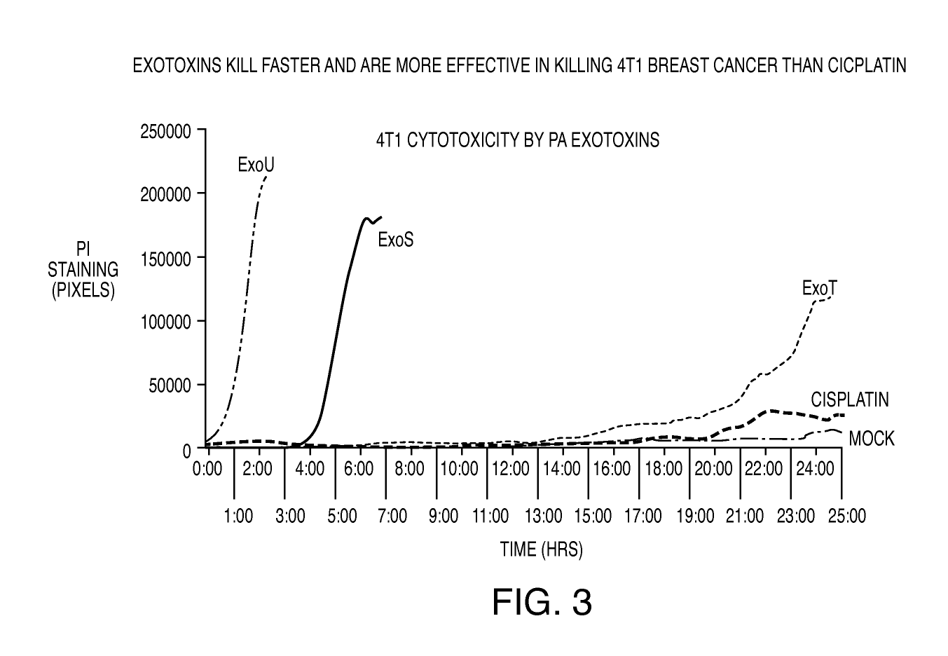

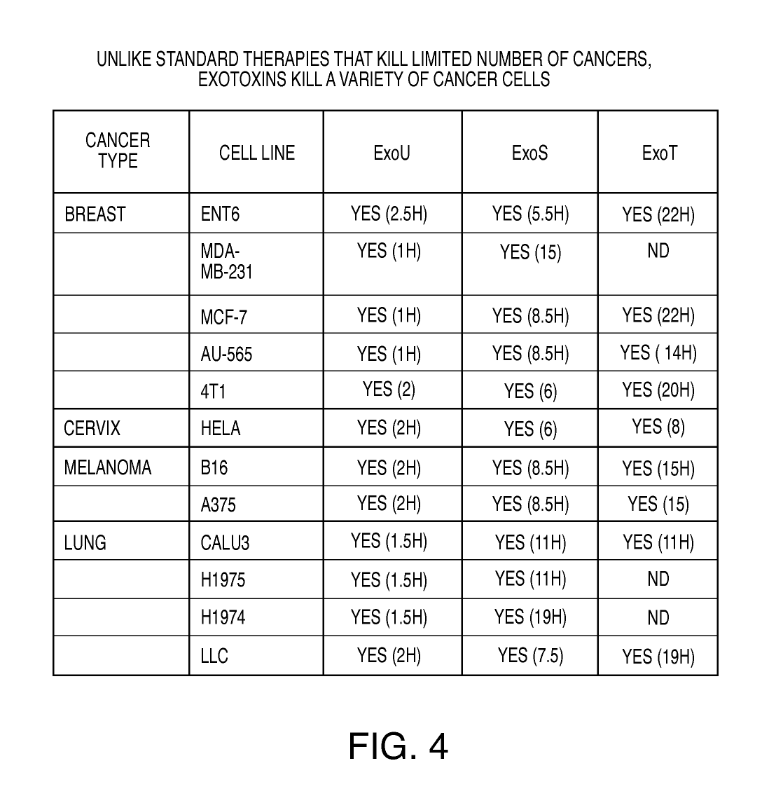

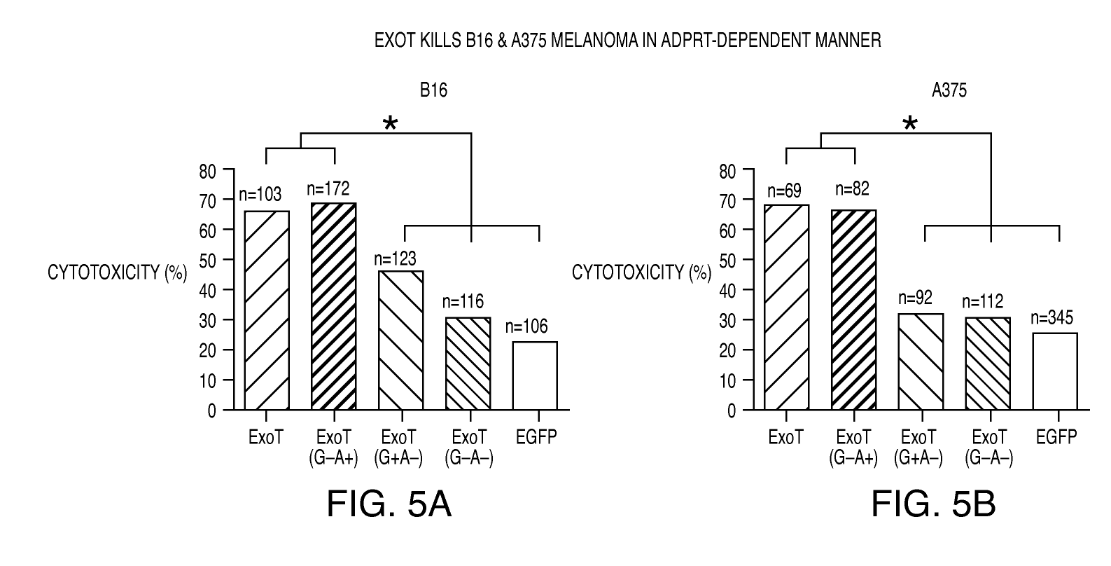

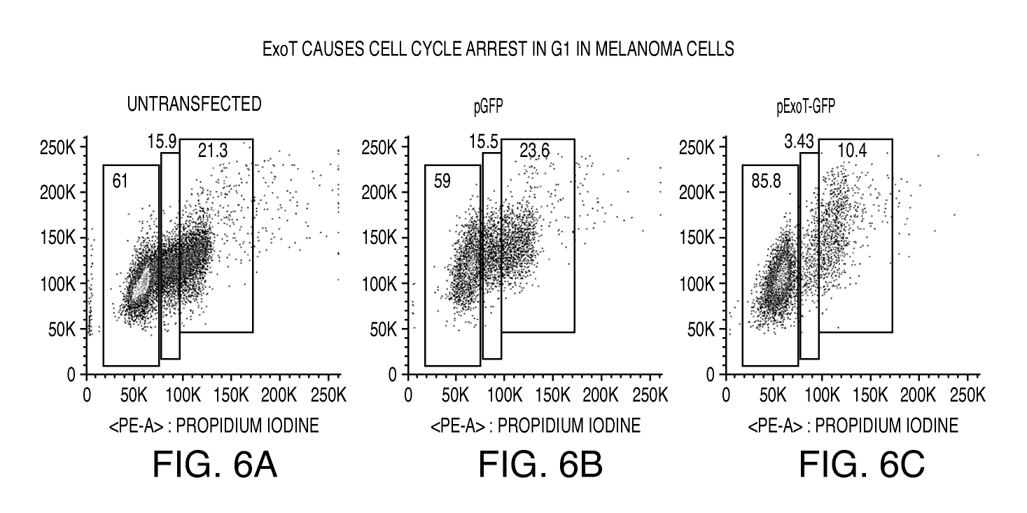

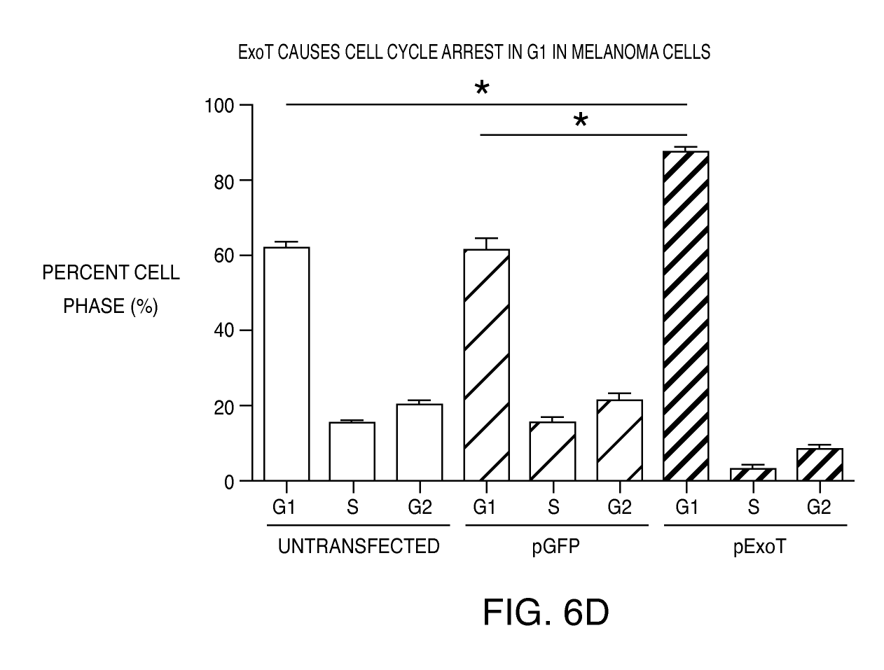

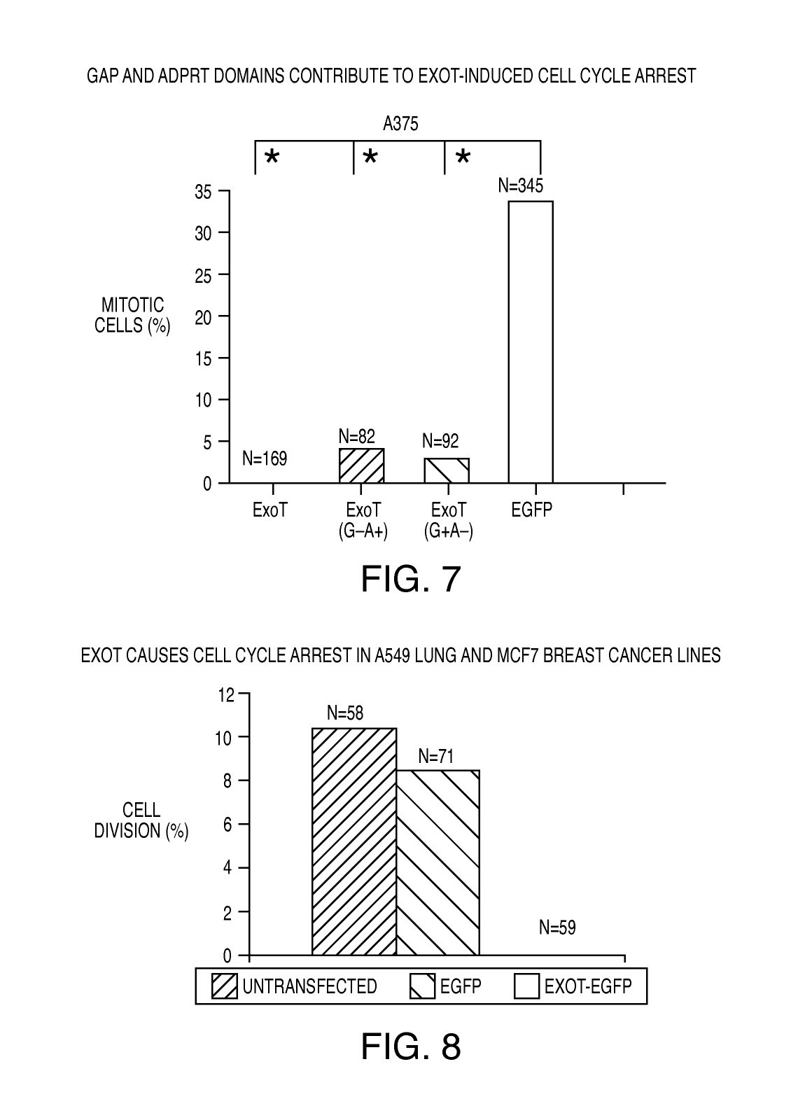

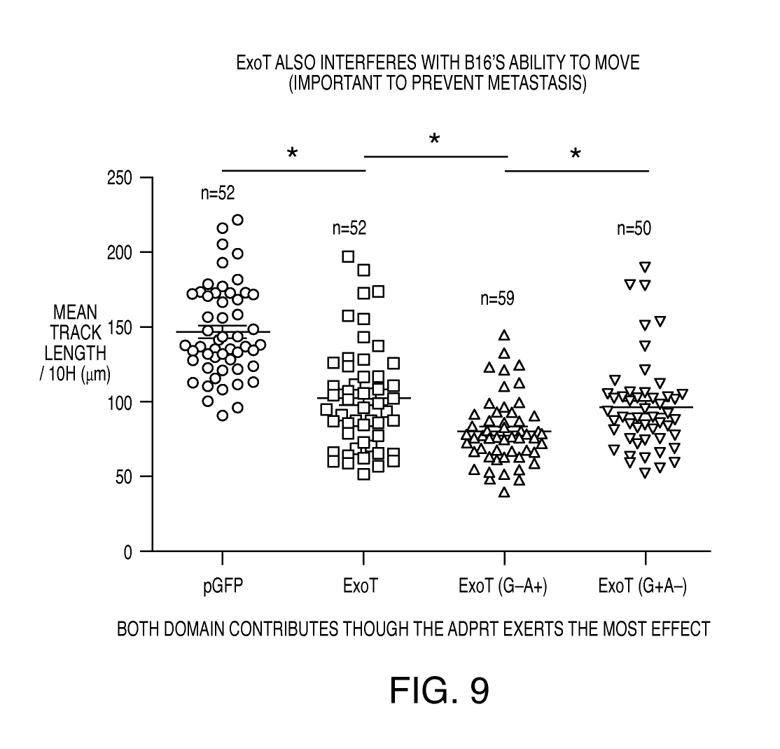

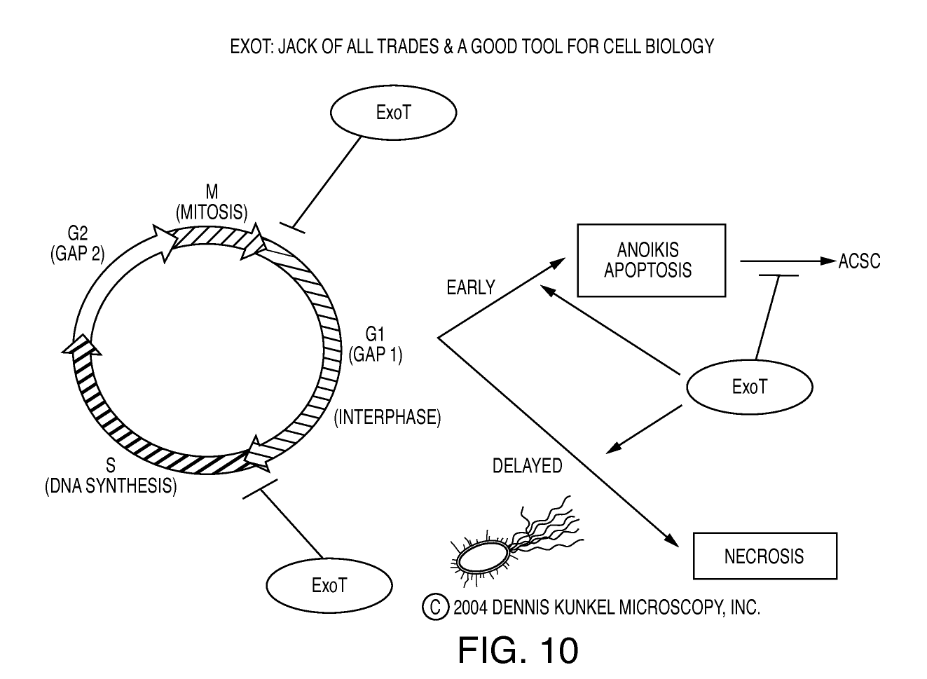

The cytotoxic effects of Pseudomonas aeruginosa exotoxins on various cancer cells are shown in FIGS. 1-10.

Further, using real time-lapse videomicroscopy, we discovered that Pseudomonas aeruginosa exotoxins are far more potent in killing various breast cancer types, as compared to standard therapy. 4T1 breast cancer cells were left untreated, treated with 50 uM cisplatin, or infected with PA expressing either ExoT, ExoS or ExoU. Cells were observed by time-lapse videomicroscopy in the presence of the cell death dye propidium iodide. The study shows that Pseudomonas aeruginosa exotoxins ExoT, ExoS, ExoU induced more potent cytotoxicity than cisplatin (at 50 μM). The exotoxins demonstrated faster kinetics and greater cytotoxicity than the chemotherapeutic drug cisplatin.

We also discovered that ExoT is effective in killing the melanoma cell lines B16 and A375. B16 cells shown here were transfected with either a GFP expressing vector or ExoT directly fused to GFP. Cells were imaged in the presence of the cell death dye propidium iodide and observed by time-lapse videomicroscopy. ExoT transfected cells undergo significant cytotoxicity relative to GFP transfected cells. A375 demonstrate a similar pattern.

Our experiments also show that ExoT is effective in killing B16 melanoma cells in vivo. B16 tumors were generated in C57B1/6 mice. ExoT-GFP was packaged with the LTX transfection reagent and injected into the tumor for 24 h. Mice were injected with the apoptotic marker dye FLIVO 1 h prior to sacrifice. Tumors were sectioned and stained with DAPI. ExoT-GFP and FLIVO colocalize indicating cell death in the tumor. Non-transfected areas are FLIVO negative, indicating living cells. Fluorescent staining confirmed that ExoT is effective in killing B16 melanoma cells in vivo.

Our experiments also show that ExoT inhibits proliferation of cancer cells. Phase time-lapse videomicroscopy was used to show B16 proliferation. B16 cells were transfected with a vector alone, which showed normal proliferation. Daughter cell proliferation was also observed. Transfection of B16 with pExoT resulted in an inhibition of cell division over the same time course as pEGFP transfected cells. A375 melanoma cells showed a similar pattern. The result shows that ExoT blocked cell division in B16 and A375 melanoma cells.

Our experiments also show that ExoT blocks entrance into S phase. B16 cells were transfected with GFP vector alone or ExoT-GFP. The cells were transfected for 24 h and then BrdU labeled for lhr. BrdU incorporation only occurs in S phase. GFP transfected cells showed BrdU labeling but there was an absence of BrdU labeling for ExoT-GFP transfected cells.

Although, bacteria have been used to deliver various toxins directly into solid tumors in vivo [44, 45], Pseudomonas aeruginosa may not a viable option for systemic delivery of ExoT because of the possibility of infection and because it does not appear to discriminate between cancerous and healthy cells/tissues.

Vaccinia virus is a member of the poxvirus family that is widely used as a eukaryotic cell expression system [46]. Vaccinia has a number of unique biological properties that make it ideally suited for delivery and amplification of transgenes within tumors. First, vaccinia has evolved mechanisms which allow it to maintain intravenous stability and spread to distant tissues [47]. Second, vaccinia possesses an actin-based motility mechanism that allows it to rapidly spread from cell-to-cell within tissues [48]. Third, because of vaccinia's relative large size, vaccinia virions may preferentially deposit in tumors, where neovasculature has increased permeability [49]. Fourth, recombinant vaccinia virus preferentially infects cancerous cells while leaving normal tissues unharmed and it has been used successfully to deliver transgenes into tumor cells in various cancer patients [50]. Fifth, except for some minor side effects, vaccinia infection is well tolerated in cancer patients, even at extremely high infection titers (107-109 pfu) [50, 51].

Pseudomonas exotoxins are likely to be far more effective in combatting metastatic tumors because of: (i) their high cytotoxicities, (ii) broad spectrum of activity toward various cancer cell lines, (iii) reduced possibility of resistance in tumor cells due to their multiple cellular targets, (iv) their potential to induce immunogenic cytotoxicity which may activate tumor-specific immune responses, needed for a durable and systemic response against tumor. (v) In addition, exotoxin T can also block the apoptotic compensatory proliferation signaling and induce G1 cell cycle arrest in tumor cells that are resistant to its cytotoxicity, thus making this toxin particularly useful in combination therapy.

### REFERENCES

1. American Cancer Society. Cancer facts and Figures 2007. Atlantic: American Cancer Society, 2007.

2. Jemal, A., et al., Cancer statistics, 2010. CA Cancer J Clin, 2010. 60(5): p. 277-300.

3. Gonzalez-Angulo, A. M., F. Morales-Vasquez, and G. N. Hortobagyi, Overview of resistance to systemic therapy in patients with breast cancer. Adv Exp Med Biol, 2007. 608: p. 1-22.

4. Gottesman, M. M., Mechanisms of cancer drug resistance. Annu Rev Med, 2002. 53: p. 615-27.

5. Calcagno, A. M. and S. V. Ambudkar, Molecular mechanisms of drug resistance in single-step and multi-step drug-selected cancer cells. Methods Mol Biol, 2010. 596: p. 77-93.

6. Kreitman, R. J., Immunotoxins for targeted cancer therapy. AAPS J, 2006. 8(3): p. E532-51.

7. Kawakami, K., et al., Targeted anticancer immunotoxins and cytotoxic agents with direct killing moieties. Scientific World Journal, 2006. 6: p. 781-90.

8. Kreitman, R. J., Recombinant immunotoxins for the treatment of chemoresistant hematologic malignancies. Curr Pharm Des, 2009. 15(23): p. 2652-64.

9. Jorgensen, R., et al., The nature and character of the transition state for the ADP-ribosyltransferase reaction. EMBO Rep, 2008. 9(8): p. 802-9.

10. Foley, B. T., J. M. Moehring, and T. J. Moehring, Mutations in the elongation factor 2 gene which confer resistance to diphtheria toxin and Pseudomonas exotoxin A. Genetic and biochemical analyses. The Journal of biological chemistry, 1995. 270(39): p. 23218-25.

11. Coley, H. M., Mechanisms and strategies to overcome chemotherapy resistance in metastatic breast cancer. Cancer Treat Rev, 2008. 34(4): p. 378-90.

12. Baguley, B. C., Multiple drug resistance mechanisms in cancer. Mol Biotechnol, 2010. 46(3): p. 308-16.

13. Dalton, W. S., Mechanisms of drug resistance in breast cancer. Semin Oncol, 1990. 17(4 Suppl 7): p. 37-9.

14. Wilson, T. R., P. G. Johnston, and D. B. Longley, Anti-apoptotic mechanisms of drug resistance in cancer. Curr Cancer Drug Targets, 2009. 9(3): p. 307-19.

15. Dunne, W. M., Jr., Bacterial adhesion: seen any good biofilms lately? Clin Microbiol Rev, 2002. 15(2): p. 155-66.

16. Teng, M. W., et al., Immune-mediated dormancy: an equilibrium with cancer. J Leukoc Biol, 2008. 84(4): p. 988-93.

17. Zitvogel, L., et al., The anticancer immune response: indispensable for therapeutic success? J Clin Invest, 2008. 118(6): p. 1991-2001.

18. Baek, S., et al., Combination therapy of renal cell carcinoma or breast cancer patients with dendritic cell vaccine and IL-2: results from a phase I/II trial. J Transl Med, 2011. 9: p. 178.

19. Zitvogel, L., et al , [Immunological aspects of anticancer chemotherapy]. Bull Acad Natl Med, 2008. 192(7): p. 1469-87; discussion 1487-9.

20. Mahmoud, S. M., et al., Tumor-infiltrating CD8+ lymphocytes predict clinical outcome in breast cancer. J Clin Oncol, 2011. 29(15): p. 1949-55.

21. Cummings, J., et al., Apoptosis pathway-targeted drugs—from the bench to the clinic. Biochim Biophys Acta, 2004. 1705(1): p. 53-66.

22. Reed, J. C., Drug insight: cancer therapy strategies based on restoration of endogenous cell death mechanisms. Nat Clin Pract Oncol, 2006. 3(7): p. 388-98.

23. Rahmani, M., et al., The kinase inhibitor sorafenib induces cell death through a process involving induction of endoplasmic reticulum stress. Mol Cell Biol, 2007. 27(15): p. 5499-513.

24. Schattenberg, J. M., M. Schuchmann, and P. R. Galle, Cell death and hepatocarcinogenesis: Dysregulation of apoptosis signaling pathways. J Gastroenterol Hepatol, 2011.26 Suppl 1: p. 213-9.

25. Fan, Y. and A. Bergmann, Apoptosis-induced compensatory proliferation. The Cell is dead. Long live the Cell! Trends Cell Biol, 2008. 18(10): p. 467-73.

26. Fan, Y. and A. Bergmann, Distinct mechanisms of apoptosis-induced compensatory proliferation in proliferating and differentiating tissues in the Drosophila eye. Dev Cell, 2008. 14(3): p. 399-410.

27. Huh, J. R., M. Guo, and B. A. Hay, Compensatory proliferation induced by cell death in the Drosophila wing disc requires activity of the apical cell death caspase Dronc in a nonapoptotic role. Curr Biol, 2004. 14(14): p. 1262-6.

28. Valentin-Vega, Y. A., H. Okano, and G. Lozano, The intestinal epithelium compensates for p53-mediated cell death and guarantees organismal survival. Cell Death Differ, 2008. 15(11): p. 1772-81.

29. Ryoo, H. D., T. Gorenc, and H. Steller, Apoptotic cells can induce compensatory cell proliferation through the JNK and the Wingless signaling pathways. Dev Cell, 2004. 7(4): p. 491-501.

30. Nazarewicz, R. R., et al., Tamoxifen induces oxidative stress and mitochondrial apoptosis via stimulating mitochondrial nitric oxide synthase. Cancer Res, 2007. 67(3): p. 1282-90.

31. Philchenkov, A., Caspases: potential targets for regulating cell death. J Cell Mol Med, 2004. 8(4): p. 432-44.

32. Fulda, S. and K. M. Debatin, Extrinsic versus intrinsic apoptosis pathways in anticancer chemotherapy. Oncogene, 2006. 25(34): p. 4798-811.

33. Maresso, A. W., M. R. Baldwin, and J. T. Barbieri, Ezrin/radixin/moesin proteins are high affinity targets for ADP-ribosylation by Pseudomonas aeruginosa ExoS. J Biol Chem, 2004. 279(37): p. 38402-8.

34. Engel, J. and P. Balachandran, Role of Pseudomonas aeruginosa type III effectors in disease. Curr Opin Microbiol, 2009. 12(1): p. 61-6.

35. Fornier, M. N., Approved agents for metastatic breast cancer. Semin Oncol, 2011. 38 Suppl 2: p. S3-10.

36. Burstein, H. J., Novel agents and future directions for refractory breast cancer. Semin Oncol, 2011. 38 Suppl 2: p. S17-24.

37. Fink, S. L. and B. T. Cookson, Apoptosis, pyroptosis, and necrosis: mechanistic description of dead and dying eukaryotic cells. Infect Immun, 2005. 73(4): p. 1907-16.

38. Krysko, D. V., et al., Apoptosis and necrosis: detection, discrimination and phagocytosis. Methods, 2008. 44(3): p. 205-21.

39. Cuzick, A., et al., The type III pseudomonal exotoxin U activates the c-Jun NH2-terminal kinase pathway and increases human epithelial interleukin-8 production. Infect Immun, 2006. 74(7): p. 4104-13.

40. Lafferty, E. I., S. T. Qureshi, and M. Schnare, The role of toll-like receptors in acute and chronic lung inflammation. J Inflamm (Lond), 2010. 7: p. 57.

41. Li, F., et al., Apoptotic cells activate the “phoenix rising” pathway to promote wound healing and tissue regeneration. Sci Signal, 2010. 3(110): p. ra13.

42. Sang, M., et al., Melanoma-associated antigen genes—an update. Cancer Lett, 2011. 302(2): p. 85-90.

43. Sivendran, S., et al., Herpes simplex virus oncolytic vaccine therapy in melanoma. Expert Opin Biol Ther, 2010. 10(7): p. 1145-53.

44. Lemmon, M. J., et al., Anaerobic bacteria as a gene delivery system that is controlled by the tumor microenvironment. Gene Ther, 1997. 4(8): p. 791-6.

45. Fox, M. E., et al., Anaerobic bacteria as a delivery system for cancer gene therapy: in vitro activation of 5-fluorocytosine by genetically engineered clostridia. Gene Ther, 1996. 3(2): p. 173-8.

46. Earl, P. L., et al., Generation of recombinant vaccinia virus, in Current Protocols in Molecular Biology, V. B. Chanda, Editor 1998, Wiley: New York, p. 16171-161719.

47. Vanderplasschen, A., et al., Extracellular enveloped vaccinia virus is resistant to complement because of incorporation of host complement control proteins into its envelope. Proceedings of the National Academy of Sciences of the United States of America, 1998. 95(13): p. 7544-9.

48. Smith, G. L., B. J. Murphy, and M. Law, Vaccinia virus motility. Annu Rev Microbiol, 2003. 57: p. 323-42.

49. Kim, D. H. and S. H. Thorne, Targeted and armed oncolytic poxviruses: a novel multi-mechanistic therapeutic class for cancer. Nat Rev Cancer, 2009. 9(1): p. 64-71.

50. Breitbach, C. J., et al., Intravenous delivery of a multi-mechanistic cancer-targeted oncolytic poxvirus in humans. Nature, 2011. 477(7362): p. 99-102.

51. Park, B. H., et al., Use of a targeted oncolytic poxvirus, JX-594, in patients with refractory primary or metastatic liver cancer: a phase I trial. Lancet Oncol, 2008. 9(6): p. 533-42.

52. Taylor, B. N., et al., Noncationic peptides obtained from azurin preferentially enter cancer cells. Cancer Res, 2009. 69(2): p. 537-46.

The specification is most thoroughly understood in light of the teachings of the references cited within the specification. The embodiments within the specification provide an illustration of embodiments of the invention and should not be construed to limit the scope of the invention. The skilled artisan readily recognizes that many other embodiments are encompassed by the invention. All publications and patents and NCBI Entrez or gene ID sequences cited in this disclosure are incorporated by reference in their entirety. To the extent the material incorporated by reference contradicts or is inconsistent with this specification, the specification will supersede any such material. The citation of any references herein is not an admission that such references are prior art to the present invention.

Those skilled in the art will recognize, or be able to ascertain using no more than routine experimentation, many equivalents to the specific embodiments of the invention described herein. Such equivalents are intended to be encompassed by the following embodiments.

### Sequences

(nucleotide sequence of ExoU)
SEQ ID NO: 1
ATGCATATCCAATCGTTGGGGGCTACTGCCTCCTCGCTGAATCAGGAGCCTGTCGAAACCCC
GTCGCAGGCAGCGCATAAGTCCGCCAGCTTGCGTCAGGAACCTTCAGGGCAAGGTCTCGGGG
TTGCCCTAAAGAGCACGCCGGGAATACTTTCCGGGAAGTTGCCGGAAAGCGTTAGCGACGTG
CGTTTCAGCAGTCCCCAAGGGCAAGGGGAGTCCCGTACTCTGACTGACTCGGCAGGGCCGCG
GCAGATCACTCTGCGCCAGTTTGAGAACGGAGTCACCGAGCTACAGCTCAGTCGGCCACCAT
TGACCAGTCTGGTCCTAAGCGGCGGTGGTGCCAAAGGTGCGGCATACCCGGGAGCAATGCTG
GCGCTAGAAGAGAAAGGCATGCTCGATGGCATCCGCAGCATGTCCGGTTCGTCCGCTGGCGG
CATCACCGCCGCCCTTTTGGCCTCAGGTATGAGCCCGGCGGCGTTCAAGACCCTTTCCGACA
AGATGGATCTTATTTCGCTGCTCGACAGCTCGAACAAGAAGCTGAAGCTGTTCCAACACATT
AGCAGCGAGATCGGCGCATCGCTGAAAAAGGGCTTGGGCAACAAGATCGGCGGCTTCTCTGA
GTTGCTGCTCAATGTACTCCCACGCATAGATTCGCGGGCTGAGCCCCTAGAACGCCTATTGC
GCGACGAGACACGCAAGGCCGTGCTCGGACAGATCGCTACGCATCCAGAGGTTGCACGCCAG
CCGACCGTTGCCGCCATCGCCAGCAGATTGCAGTCCGGCTCCGGAGTCACCTTTGGCGATCT
AGATCGGTTGAGTGCTTACATTCCCCAGATTAAGACGCTGAACATCACAGGTACGGCCATGT
TCGAGGGGCGTCCGCAATTAGTGGTGTTCAATGCCAGCCACACACCGGATCTGGAGGTCGCC
CAGGCGGCACATATCTCCGGTTCCTTCCCAGGAGTGTTCCAGAAGGTCAGCTTGAGTGATCA
GCCGTACCAGGCCGGCGTAGAGTGGACAGAATTCCAGGATGGCGGGGTGATGATTAACGTGC
CGGTCCCTGAGATGATCGACAAGAATTTTGACAGCGGGCCACTGCGGCGCAACGACAACCTG
ATCCTTGAGTTCGAGGGCGAAGCTGGGGAGGTAGCGCCCGACCGAGGTACTAGGGGCGGCGC
GCTCAAGGGCTGGGTCGTCGGGGTGCCTGCCCTGCAGGCGCGCGAAATGCTGCAGCTCGAGG
GCCTGGAGGAATTGCGCGAGCAAACCGTTGTGGTGCCGTTGAAGAGCGAGCGCGGTGATTTC
AGTGGCATGCTCGGTGGCACCTTGAACTTCACCATGCCGGACGAGATCAAGGCGCATCTTCA
GGAGCGCCTCCAGGAGCGAGTCGGTGAACATCTGGAGAAACGTCTTCAGGCTTCAGAGCGTC
ATACCTTCGCTTCTCTCGACGAGGCGCTGCTGGCACTTGATGACAGTATGCTCACCAGTGTT
GCTCAACAGAACCCGGAGATCACAGACGGGGCGGTGGCTTTTCGCCAGAAGGCGCGGGATGC
GTTCACCGAGCTGACTGTCGCTATCGTTAGCGCCAATGGCTTGGCGGGTAGGCTCAAGTTGG
ACGAGGCTATGCGCTCCGCTCTTCAGCGACTCGATGCGCTGGCAGATACTCCGGAACGCCTA
GCATGGTTGGCAGCTGAGTTGAACCATGCTGATAACGTTGATCATCAGCAGTTACTCGATGC
CATGCGCGGGCAGACGGTGCAGTCGCCGGTGCTCGCCGCTGCGTTAGCAGAGGCGCAGCGCC
GCAAAGTGGCGGTTATTGCCGAGAACATTCGTAAGGAAGTTATCTTCCCCTCTCTGTATCGC
CCTGGCCAGCCGGATTCCAACGTAGCTCTGTTACGTCGGGCGGAGGAGCAGCTACGGCATGC
CACCAGTCCGGCGGAAATCAATCAAGCGCTGAACGATATCGTCGACAACTACTCGGCACGAG
GCTTCCTGCGTTTCGGCAAACCCTTGAGTTCGACTACCGTTGAGATGGCTAAGGCTTGGCGG
AATAAGGAGTTCACATGA
(Amino acid sequence of ExoU)
SEQ ID NO: 2
MHIQSLGATASSLNQEPVETPSQAAHKSASLRQEPSGQGLGVALKSTPGILSGKLPESVSDV
RFSSPQGQGESRTLTDSAGPRQITLRQFENGVTELQLSRPPLTSLVLSGGGAKGAAYPGAML
ALEEKGMLDGIRSMSGSSAGGITAALLASGMSPAAFKTLSDKMDLISLLDSSNKKLKLFQHI
SSEIGASLKKGLGNKIGGFSELLLNVLPRIDSRAEPLERLLRDETRKAVLGQIATHPEVARQ
PTVAAIASRLQSGSGVTFGDLDRLSAYIPQIKTLNITGTAMFEGRPQLVVFNASHTPDLEVA
QAAHISGSFPGVFQKVSLSDQPYQAGVEWTEFQDGGVMINVPVPEMIDKNFDSGPLRRNDNL
ILEFEGEAGEVAPDRGTRGGALKGWVVGVPALQAREMLQLEGLEELREQTVVVPLKSERGDF
SGMLGGTLNFTMPDEIKAHLQERLQERVGEHLEKRLQASERHTFASLDEALLALDDSMLTSV
AQQNPEITDGAVAFRQKARDAFTELTVAIVSANGLAGRLKLDEAMRSALQRLDALADTPERL
AWLAAELNHADNVDHQQLLDAMRGQTVQSPVLAAALAEAQRRKVAVIAENIRKEVIFPSLYR
PGQPDSNVALLRRAEEQLRHATSPAEINQALNDIVDNYSARGFLRFGKPLSSTTVEMAKAWR
NKEFT*
(nucleotide sequence of ExoT)
SEQ ID NO: 3
ATGCATATTCAATCATCTCAGCAGAACCCGTCTTTCGTGGCTGAGTTGAGCCAGGCCGTGGC
CGGGCGCCTGGGACAGGTCGAGGCCCGCCAGGTGGCCACTCCCCGGGAGGCGCAACAACTGG
CCCAGCGCCAGGAAGCACCGAAGGGCGAGGGCCTGCTCTCCCGCCTGGGGGCTGCCCTCGCG
CGTCCCTTCGTGGCGATCATCGAGTGGCTGGGCAAACTGCTGGGGAGCCGTGCCCACGCCGC
CACCCAGGCGCCGCTCTCCCGTCAGGACGCGCCGCCTGCCGCCAGTCTCTCTGCCGCCGAGA
TCAAGCAGATGATGCTGCAAAAGGCACTGCCCCTGACCTTGGGCGGACTTGGCAAGGCGAGC
GAGCTGGCGACTTTGACAGCGGAGAGGCTGGCGAAGGATCACACGCGCCTGGCCAGCGGCGA
CGGCGCTCTGCGATCGCTGGCCACCGCCCTGGTCGGGATTCGCGATGGCAGCCGGATCGAGG
CTTCCCGTACCCAGGCTGCCCGCCTGCTCGAACAGAGCGTTGGGGGGATCGCGCTGCAACAG
TGGGGGACCGCGGGCGGTGCCGCCAGCCAGCATGTACTCAGCGCAAGCCCGGAGCAACTGCG
CGAAATCGCCGTCCAACTGCATGCGGTAATGGACAAGGTCGCCCTGTTGCGCCACGCGGTAG
AGAGCGAGGTAAAGGGCGAGCCTGTCGACAAGGCGCTGGCGGATGGCCTGGTGGAGCACTTC
GGGCTGGAGGCGGAGCAGTACCTCGGCGAACACCCGGACGGGCCGTACAGCGATGCCGAGGT
GATGGCGCTCGGTCTCTATACCAACGGCGAGTACCAGCACCTGAATCGGTCCCTGCGTCAGG
GACGGGAGCTGGATGCGGGCCAGGCGTTGATCGACCGGGGCATGTCTGCCGCGTTCGAAAAG
AGCGGACCGGCTGAACAGGTCGTGAAGACCTTCCGCGGCACCCAGGGCAGGGATGCCTTCGA
GGCGGTGAAAGAGGGCCAGGTCGGCCACGACGCCGGCTATCTCTCCACCTCCCGGGACCCCG
GCGTTGCCAGGAGCTTCGCGGGCCAGGGCACGATAACCACCCTGTTCGGCAGATCCGGGATC
GATGTCAGCGAGATATCGATCGAGGGCGATGAGCAGGAGATCCTCTACGACAAGGGGACCGA
CATGCGCGTGCTTCTCAGTGCCAAGGATGGGCAGGGTGTGACCCGTCGGGTGCTCGAAGAGG
CCACGCTGGGGGAACGGAGCGGCCACGGCGAGGGACTGCTCGATGCCCTGGACCTGGCAACC
GGGACGGATCGTTCAGGCAAGCCCCAGGAACAGGACCTGCGCCTGAGAATGCGCGGCCTCGA
CCTGGCCTGA
(Amino acid sequence of ExoT)
SEQ ID NO: 4
MHIQSSQQNPSFVAELSQAVAGRLGQVEARQVATPREAQQLAQRQEAPKGEGLLSRLGAALA
RPFVAIIEWLGKLLGSRAHAATQAPLSRQDAPPAASLSAAEIKQMMLQKALPLTLGGLGKAS
ELATLTAERLAKDHTRLASGDGALRSLATALVGIRDGSRIEASRTQAARLLEQSVGGIALQQ
WGTAGGAASQHVLSASPEQLREIAVQLHAVMDKVALLRHAVESEVKGEPVDKALADGLVEHF
GLEAEQYLGEHPDGPYSDAEVMALGLYTNGEYQHLNRSLRQGRELDAGQALIDRGMSAAFEK
SGPAEQVVKTFRGTQGRDAFEAVKEGQVGHDAGYLSTSRDPGVARSFAGQGTITTLFGRSGI
DVSEISIEGDEQEILYDKGTDMRVLLSAKDGQGVTRRVLEEATLGERSGHGEGLLDALDLAT
GTDRSGKPQEQDLRLRMRGLDLA*
(nucleotide sequence of ExoS)
SEQ ID NO: 5
ATGCATATTCAATCGCTCCAGCAGAGTCCGTCTTTCGCCGTCGAATTGCACCAGGCCGCCAG
TGGGCGTTTGGGACAGATTGAGGCCCGCCAGGTCGCCACCCCCAGTGAAGCGCAGCAGTTGG
CCCAGCGCCAGGACGCGCCGAAGGGTGAGGGGCTGCTCGCTCGCCTGGGCGCGGCGCTCGTG
CGTCCGTTCGTGGCGATCATGGACTGGCTGGGCAAACTGTTGGGCTCCCACGCCCGCACCGG
GCCGCAGCCCAGTCAGGACGCGCAGCCTGCGGTCATGTCCTCGGCCGTCGTGTTCAAGCAGA
TGGTGCTGCAGCAGGCATTGCCCATGACCTTGAAGGGACTCGACAAGGCGAGCGAGCTGGCG
ACCCTGACACCGGAAGGACTGGCCCGGGAGCACTCCCGCCTGGCCAGCGGAGATGGGGCGCT
GCGTTCGCTGAGCACCGCCTTGGCCGGCATTCGTGCCGGCAGCCAGGTCGAGGAGTCCCGTA
TCCAGGCTGGCCGCCTGCTCGAACGGAGCATCGGCGGGATCGCGCTGCAGCAGTGGGGCACC
ACCGGCGGTGCCGCGAGTCAACTGGTGCTCGACGCAAGCCCGGAACTGCGGCGCGAAATCAC
CGACCAGTTGCATCAGGTAATGAGCGAGGTCGCACTGTTGCGCCAAGCGGTAGAGAGCGAGG
TCAGCAGAGTATCGGCCGACAAGGCGCTGGCGGATGGCCTGGTGAAGCGGTTCGGGGCGGAT
GCGGAAAAGTACCTGGGCAGACAGCCTGGTGGCATCCACAGTGACGCCGAAGTGATGGCGCT
TGGTCTCTACACCGGCATTCACTACGCGGACCTGAATCGCGCTCTGCGTCAGGGGCAGGAGC
TGGATGCGGGACAAAAGCTGATCGACCAAGGTATGTCCGCGGCCTTCGAGAAGAGCGGACAG
GCTGAACAGGTAGTGAAGACTTTCCGTGGCACCCGTGGCGGGGATGCCTTCAACGCAGTGGA
AGAGGGCAAGGTTGGCCACGACGACGGCTATCTCTCCACCTCCCTGAACCCCGGTGTCGCGA
GGAGCTTCGGGCAGGGCACGATATCCACCGTGTTCGGCAGGTCCGGAATCGATGTCAGCGGG
ATATCGAACTACAAGAATGAAAAAGAGATTCTCTATAACAAAGAAACCGACATGCGCGTGCT
GCTGAGCGCCAGCGATGAGCAGGGAGTGACCCGCCGGGTTCTCGAAGAGGCGGCCCTGGGGG
AGCAGAGTGGCCATAGCCAGGGACTGCTCGATGCTCTCGACCTGGCAAGCAAACCGGAACGT
TCAGGCGAGGTCCAGGAACAGGATGTACGCCTGAGGATGCGCGGCCTTGATCTGGCCTGA
(Amino acid sequence of ExoS)
SEQ ID NO: 6
MHIQSLQQSPSFAVELHQAASGRLGQIEARQVATPSEAQQLAQRQDAPKGEGLLARLGAALV
RPFVAIMDWLGKLLGSHARTGPQPSQDAQPAVMSSAVVFKQMVLQQALPMTLKGLDKASELA
TLTPEGLAREHSRLASGDGALRSLSTALAGIRAGSQVEESRIQAGRLLERSIGGIALQQWGT
TGGAASQLVLDASPELRREITDQLHQVMSEVALLRQAVESEVSRVSADKALADGLVKRFGAD
AEKYLGRQPGGIHSDAEVMALGLYTGIHYADLNRALRQGQELDAGQKLIDQGMSAAFEKSGQ
AEQVVKTFRGTRGGDAFNAVEEGKVGHDDGYLSTSLNPGVARSFGQGTISTVFGRSGIDVSG
ISNYKNEKEILYNKETDMRVLLSASDEQGVTRRVLEEAALGEQSGHSQGLLDALDLASKPER
SGEVQEQDVRLRMRGLDLA*

1. A recombinant viral vector comprising a nucleic acid sequence encoding a Pseudomonas aeruginosa exotoxin.

2. The recombinant viral vector of claim 1, wherein said exotoxin is selected from the group consisting of: S (ExoS), exotoxin T (ExoT), exotoxin U (ExoU), a cytotoxic fragment thereof, and a combination thereof.

3. The recombinant viral vector of claim 2, wherein said ExoU comprises an amino acid sequence that is at least 85% identical to SEQ ID NO:2.

4. The recombinant viral vector of claim 2, wherein said ExoT comprises an amino acid sequence that is at least 85% identical to SEQ ID NO:4.

5. The recombinant viral vector of claim 2, wherein said ExoS comprises an amino acid sequence that is at least 85% identical to SEQ ID NO:6.

6. The recombinant viral vector of claim 1, wherein said viral vector is a: Vaccinia virus vector, Adenovirus vector, HSV vector, Newcastle Disease Virus vector, Reovirus vector, Coxsackievirus vector, or Senneca Valley Virus vector.

7. The recombinant viral vector of claim 1, wherein said viral vector is a Vaccinia virus vector.

8. A method of treating cancer, comprising: administering to a subject in need thereof a therapeutically effective amount of a recombinant viral vector, wherein said viral vector comprises a nucleic acid sequence encoding a Pseudomonas aeruginosa exotoxin.

9. The method of claim 8, wherein said exotoxin is selected from the group consisting of: S (ExoS), exotoxin T (ExoT), exotoxin U (ExoU), a cytotoxic fragment thereof, and a combination thereof.

10. The method of claim 9, wherein said ExoU comprises an amino acid sequence that is at least 85% identical to SEQ ID NO:2.

11. The method of claim 9, wherein said ExoT comprises an amino acid sequence that is at least 85% identical to SEQ ID NO:4.

12. The method of claim 9, wherein said ExoS comprises an amino acid sequence that is at least 85% identical to SE Q ID NO:6.

13. The method of claim 9, wherein said viral vector is a: Vaccinia virus vector, Adenovirus vector, FISV vector, Newcastle Disease Virus vector, Reovirus vector, Coxsackievirus vector, or Senneca Valley Virus vector.

14. The method of claim 9, wherein said viral vector is a Vaccinia virus vector.

15. The method of claim 9, wherein said cancer is bladder cancer, breast cancer, colon cancer, kidney cancer, liver cancer, lung cancer, esophagus cancer, gall bladder cancer, ovarian cancer, pancreas cancer, stomach cancer, cervical cancer, thyroid cancer, prostate cancer, testicular cancer, skin cancer, leukemia, B-cell lymphoma, T-cell lymphoma, Hodgkins lymphoma, non-Hodgkins lymphoma, hairy cell lymphoma, Burkett's lymphoma, fibrosarcoma, rhabdomyosarcoma, astrocytoma, neuroblastoma, gliorna and schwannomas, melanoma, seminoma, teratocarcinoma, osteosarcoma, xenoderoma pigmentosurn, keratoctanthoma, thyroid follicular cancer, or Kaposi's sarcoma.

16. The method of claim 9, wherein said cancer is breast cancer.

17. The method of claim 9, further comprising administering to said subject another chemotherapeutic agent.

18. A recombinant virus comprising the recombinant viral vector of claim 1.

19. A pharmaceutical composition comprising the recombinant viral vector of claim 1 and/or the recombinant virus of claim 18.

20. The method of claim 8, wherein the recombinant viral vector is administered in the form of a recombinant virus.

21. A method of making a recombinant viral vector for delivering a Pseudomonas aeruginosa exotoxin to a host cell, comprising: (i) providing a viral vector, (ii) inserting a nucleic acid sequence encoding said Pseudomonas aeruginosa exotoxin into said viral vector, wherein the exotoxin-coding sequence is operably linked to an expression control sequence, such that said exotoxin is expressed in said host cell.
